(19) United States
(12) Patent Application Publication
(10) Pub. No.: US 2022/0087849 A1
(43) Pub. Date: Mar. 24, 2022

(54) SYSTEMS AND METHODS FOR PREVENTING AND MANAGING SORES, ULCERS, AND WOUNDS

(71) Applicant: THE GENERAL HOSPITAL CORPORATION, Boston, MA (US)

(72) Inventors: Joe C. Manso, Boston, MA (US); Christopher W. Digiovanni, Boston, MA (US); Gregory Waryasz, Boston, MA (US); Daniel Guss, Boston, MA (US); Bart Lubberts, Boston, MA (US)

(21) Appl. No.: 17/425,526

(22) PCT Filed: Jan. 23, 2020
(86) PCT No.: PCT/US2020/014752

§ 371 Date: Jul. 23, 2021

Related U.S. Application Data
(60) Provisional application No. 62/795,840, filed on Jan. 23, 2019.

(51) Int. Cl. A61F 5/01

(52) U.S. Cl. CPC A61F 5/0195; A61F 2005/0179; A61F 2005/0172; A61F 5/0127

(57) ABSTRACT

A system and method is provided for managing sores, ulcers, and wounds. An orthopedic boot comprises, a sole; a support member connected to and upwardly extending from the sole, the support member being adapted to secure the boot to a foot of a subject; and a plurality of support columns removably coupled to and upwardly extending from the sole.

### CROSS-REFERENCE TO RELATED APPLICATIONS

This application is based on, claims the benefit of, and claims priority to U.S. Provisional Application No. 62/795,840, filed Jan. 23, 2019, which is hereby incorporated by reference herein in its entirety for all purposes.

### STATEMENT REGARDING FEDERALLY SPONSORED RESEARCH

Not applicable.

### BACKGROUND

The formation and treatment of pressure sores and ulcers continue to be problematic for patients following surgery. Generally, pressure ulcers (and sores) are caused by frequent reoccurring pressure or friction to a region of soft tissue or skin, resulting in impeded blood flow, and thus the formation of the sore. Depending on a number of factors (such as degree of immobility), the severity of the pressure sore can range from stage I being the least severe, to stage IV being the most severe. Patients with chronic diseases that impact blood flow are especially prone to pressure ulcers. For example, those with diabetes tend to already have decreased blood flow to their extremities. Thus, the immobilization of these extremities following orthopedic surgery only enhances the conditions required for the formation of pressure sores. Although individuals with chronic diseases may be more likely to develop pressure sores, individuals free of blood perfusion diseases can still develop pressure sores. In fact, sedentary individuals and those with appendage immobilizations (such as following a surgery) while generally being in good health, are still susceptible to pressure sore formation (e.g., at least due to the inherent lack of sufficient blood flow to the region).

While pressure ulcers can be treated, sometimes pressure ulcers can result in complications such as infections, bleeding, swelling, etc., all of which increase the strain on health care professionals (e.g., increased time from nurses and physicians, and increased expenses). Thus, not surprisingly pressure ulcers (including diabetic related pressure ulcers) impose significant and unwanted costs to the healthcare system.

Thus, it would be desirable to have improved systems and methods for preventing and managing sores, ulcers, and wounds.

### SUMMARY OF THE DISCLOSURE

In accordance with some embodiments of the disclosed subject matter, systems, and methods, for preventing and managing sores, ulcers, and wounds are provided.

Any of the features, components, elements etc., are intended to be interchangeable between differing embodiments, according to various aspects of this disclosure.

Some embodiments of the disclosure provide an orthopedic boot comprising, a sole; a support member connected to and upwardly extending from the sole, the support member being adapted to secure the boot to a foot of a subject; and a plurality of support columns removably coupled to and upwardly extending from the sole.

In some embodiments, the sole has a first surface and an opposite second surface, the second surface being configured to contact the ground, wherein a column of the plurality of columns has an upper surface, wherein a portion of first surface of the sole and the upper surface at least partially define a void, and wherein when a foot of a subject is secured by the support member, a sore of a subject is positioned within the void.

In some embodiments, the sole has a first surface and an opposite second surface, the second surface being configured to contact the ground, wherein a first column of the plurality of columns has a first upper surface and a first height, wherein a second column of the plurality of columns has a second upper surface and a second height, wherein the first height is different than the second height, and the first surface and the second surface at least partially define a void, and wherein when a foot of a subject is secured by the support member, a sore of a subject is positioned within the void.

In some embodiments, the orthopedic boot further comprises an insole coupled to the plurality of support columns and positioned above the sole, wherein the base does not contact the insole, wherein the insole is rigid, and wherein the sole is rigid.

In some embodiments, the orthopedic boot further comprises an aperture extending through the insole, and wherein two support columns of the plurality of support columns are spaced apart from each other by a distance.

In some embodiments, the orthopedic boot further comprises a second aperture extending through the insole.

In some embodiments, the sole has a first surface and an opposite second surface, the second surface being configured to contact the ground, wherein the aperture and the first surface defines a void volume, and wherein the plurality of support columns are not positioned within the void volume.

In some embodiments, the orthopedic boot further comprises a cushioning layer coupled to the insole and positioned above the plurality of support columns, the cushioning layer comprising a memory foam, and a cushioning layer aperture extending through the cushioning layer, and wherein a portion of the aperture and a portion of the cushioning layer aperture overlap.

In some embodiments, when a foot of a subject having a sore is positioned on the insole, a perimeter of the aperture surrounds the sore to prevent contact between the orthopedic boot and the sore of the subject.

In some embodiments, the orthopedic boot further comprises a base, the base being coupled to the sole, and wherein the plurality of support columns are removably coupled to the base.

In some embodiments, the base is at least one of affixed, and removably coupled to the sole.

In some embodiments, the base has a first surface and an opposite second surface, the second surface being removably coupled to the sole, and further comprising a plurality of pegs upwardly extending from the first surface.

In some embodiments, a first support column of the plurality of support columns includes an upper surface, a lower surface, and a recess, and wherein a given peg of the plurality of pegs is inserted into the recess to secure the first support column to the base.

In some embodiments, the upper surface of the first support column is angled relative to a horizontal axis.

In some embodiments, the upper surface of the first support column is angled downwardly in a lateral direction relative to the sole.

In some embodiments, the upper surface of the first support column is angled upwardly in a lateral direction relative to the sole.

In some embodiments, the upper surface of the first support column is angled relative to a front view of the first support column.

In some embodiments, the first support column includes the recess and a second recess, and wherein a second peg of the plurality of pegs is inserted into the second recess.

In some embodiments, the lower surface has the recess and the second recess, and wherein an upper surface of the first support column is angled relative to a horizontal axis.

In some embodiments, a first support column of the plurality of support columns includes a first surface and a second surface, and further comprising a fastener coupled to the first support column, the fastener being configured to couple the first support column to the base.

In some embodiments, the first surface of the first support column has a bolt body extending therethough, and further comprising a bore extending through the base, and wherein the bolt body threadingly engages the bore to couple the first support column to the bore of the base.

In some embodiments, the orthopedic boot further comprises an insole coupled to the plurality of support columns and positioned above the sole; and a second fastener, the second fastener extending through the insole and configured to engage the second surface of the first support column to secure the insole to the first support column.

In some embodiments, the first surface of the first support column has a bore extending therethrough, and further comprising a bolt body extending from the base, and wherein the bolt body threadingly engages the bore to couple the first support column to the bolt body of the base.

In some embodiments, the base has a first surface and an opposite second surface, the base including a plurality of recesses directed into the first surface of the base.

In some embodiments, a first support column of the plurality of support columns includes a protrusion extending therefrom, and wherein the protrusion is inserted into a given recess of the plurality of recesses to secure the first support column to the base.

In some embodiments, the given recess and the protrusion have at least one of a rectangular shape, and round shape.

In some embodiments, the first support column includes a second protrusion extending therefrom, and wherein a second protrusion is inserted into another recess of the plurality of recesses to secure the first support column to the base.

In some embodiments, the column includes a first surface and a second surface, the first surface including at least one of a hook fastener, and a loop fastener.

In some embodiments, the orthopedic boot further comprises an insole coupled to the plurality of support columns and positioned above the sole, and wherein the insole includes the other of the at least one of the hook fastener and the loop fastener to removably couple the insole to the plurality of support columns.

In some embodiments, at least one of the sole, and the base has a first surface and an opposite second surface, the first surface of the base including at least one of a hook fastener, and a loop fastener.

In some embodiments, a given column of the plurality of support columns is configured to contact an adjacent column.

In some embodiments, the plurality of columns each include an upper surface and an opposite lower surface, the lower surface having the other of the at least one of the hook fastener, and the loop fastener.

In some embodiments, each of the plurality of columns includes a side surface, and wherein a first side surface for each column of a subset of the plurality of columns defines a boundary, the boundary defining a void volume, such that the plurality of columns are not positioned within the void volume.

In some embodiments, when a foot of a subject is secured by the support member, a sore of a subject is positioned within the void volume.

In some embodiments, the orthopedic boot further comprises an insole coupled to and positioned above the plurality of support columns; and a cushioning layer coupled to and positioned above the insole, the cushioning layer comprising memory foam.

In some embodiments, the support member has an interior surface defining an interior volume, the foot of the subject being configured to be received within the internal volume of the support member, and the orthopedic boot further comprises a bladder received within the interior volume, the bladder being configured to receive a source of fluid thereby expanding the bladder.

In some embodiments, the orthopedic boot further comprises an expandable shell coupled to the support member.

In some embodiments, the orthopedic boot further comprises an aperture extending through the expandable shell.

In some embodiments, the expandable shell is positioned around a portion of a heel of a subject when a foot of the subject is received within an internal volume of the support member.

In some embodiments, the orthopedic boot further comprises a plurality of pads situated within the expandable shell.

In some embodiments, the orthopedic boot further comprises a liner situated within an internal volume of the support member, the plurality of pads positioned between the liner and the expandable shell.

In some embodiments, the orthopedic boot further comprises an insole coupled to and positioned above the plurality of support columns; an aperture extending through the insole; and a tether configured to secure a toe of a subject to the insole.

In some embodiments, the tether includes a loop and an end, the end removably coupled to at least one of the insole and a first support column of the plurality of support columns by a hook and loop fastener.

In some embodiments, the loop of the tether is configured to be received around a toe of a subject, and when the end of the tether is coupled to the at least one of the insole and the first support column, the toe is secured at an angle relative to a horizontal axis.

In some embodiments, a first support column of the plurality of support columns includes a biasing member configured to exert a biasing force.

In some embodiments, the first support column includes a head, the head being coupled to the biasing member, and wherein the biasing member is a spring.

In some embodiments, the orthopedic boot further comprises a ring surrounding the support member, the ring configured to prevent removal of the support member from the subject.

In some embodiments, the orthopedic boot further comprises an insole situated above and removably coupled to the plurality of support columns.

In some embodiments, a first support column of the plurality of support columns includes a first surface and a second surface, the first surface including at least one of a hook fastener, and a loop fastener, and wherein the insole includes the other of the at least one of the hook fastener and the loop fastener to removably couple the insole to the plurality of support columns.

In some embodiments, the orthopedic boot further comprises a cushioning layer, the cushioning layer comprising a memory foam, and wherein the cushioning layer is situated above and coupled to the insole.

In some embodiments, the cushioning layer is removably coupled to the insole.

Some embodiments of the disclosure provide an orthopedic insert for use with an orthopedic boot or a shoe, the orthopedic insert comprising: a base; a plurality of support columns upwardly extending from and removably coupled to the base; an insole coupled to the plurality of support columns, the insole positioned above the base, and wherein the orthopedic insert is configured to be received within an internal volume of the at least one of the orthopedic boot, and the shoe.

In some embodiments, the orthopedic insert further comprises an aperture extending through the insole, and wherein the aperture and a surface of the base define a void volume.

In some embodiments, the plurality of support columns are not positioned within the void volume.

In some embodiments, when a foot of a subject is positioned on the insole, a sore of a subject is positioned within the void volume.

In some embodiments, the orthopedic insert further comprises a cushioning layer coupled to an situated above the insole, the cushioning layer comprising memory foam.

### BRIEF DESCRIPTIONS OF THE DRAWINGS

FIG. 1A shows a top view of an example of an orthopedic boot, according to some embodiments of the disclosure.

FIG. 1B shows a cross-sectional view of the orthopedic boot of FIG. 1A taken along line 1B-1B of FIG. 1A.

FIG. 2A shows a top view of an example of an orthopedic insert.

FIG. 2B shows a cross-sectional view of the orthopedic insert of FIG. 2A taken along line 2B-2B of FIG. 2A.

FIG. 3 an example of a base of the orthopedic insert of FIG. 2A having upwardly projecting pegs.

FIG. 4A shows a top view of an example of a peg.

FIG. 4B shows a top perspective view of the peg of FIG. 4A.

FIG. 4C shows a top view of an example of another peg.

FIG. 4D shows a top perspective view of the peg of FIG. 4C.

FIG. 5 shows an example of support columns coupled to the base of FIG. 3.

FIG. 6A shows a top view of an example of a nesting support column coupled to the base of FIG. 3.

FIG. 6B shows a top perspective view of the nesting support column of FIG. 6A.

FIG. 6C shows a bottom view of the nesting support column of FIG. 6A.

FIG. 6D shows a cross-sectional view of the nesting support column of FIG. 6A coupled to the base of FIG. 3 taken along the line 6D-6D of FIG. 6A.

FIG. 7A shows a top view of an example of a fastening support column coupled to the base of FIG. 3.

FIG. 7B shows a top perspective view of the fastening support column of FIG. 7A.

FIG. 7C shows a cross-sectional view of the fastening support column of FIG. 7A in coupling arrangement to the base of FIG. 3 taken along the line 7C-7C of FIG. 7A.

FIG. 8A shows a top view of an example of a locking support column coupled to the base of FIG. 3.

FIG. 8B shows a top exploded perspective view of a locking support column of FIG. 8A.

FIG. 8C shows a cross-sectional view of the locking support column of FIG. 8A in coupling arrangement to the base of FIG. 3 taken along the line 8C-8C of FIG. 8A.

FIG. 8D shows a top view of the locking support column of FIG. 8A without the second fastener.

FIG. 9A shows a partial top view of an example of another nesting support column in coupling arrangement to the base of FIG. 3.

FIG. 9B shows a cross-sectional view of the nesting support column in coupling arrangement with the base of FIG. 3 taken along line 9B-9B of FIG. 9A without the other support columns in fastening arrangement.

FIG. 9C shows a cross-sectional view of the nesting support column in coupling arrangement with the base of FIG. 3 taken along line 9C-9C of FIG. 9A with the other support columns in fastening arrangement.

FIG. 10A a top view of the orthopedic insert of FIGS. 2A and 2B being received within an orthopedic boot.

FIG. 10B is a cross-sectional view of the orthopedic insert of FIG. 10A received within the orthopedic boot taken along line 10B-10B of FIG. 10A.

FIG. 11A shows a top view of another orthopedic insert, showing the locations of coupled support columns.

FIG. 11B shows a top view of another orthopedic insert, showing the locations of coupled support columns.

FIG. 11C shows a top view of another orthopedic insert, showing the locations of coupled support columns.

FIG. 11D shows a top view of another orthopedic insert, showing the locations of coupled support columns.

FIG. 12 shows an example of a lower surface of the cushioning layer of the orthopedic insert of FIG. 2A.

FIG. 13A shows a diagram of a foot of a subject with labeled anatomical structures of the foot, and labeled regions that may be prone to pressure sore formation.

FIG. 13B shows the diagram of a foot of a subject of FIG. 13A with support columns in a confirmation, overlaid on the diagram.

FIG. 13C shows another diagram of a foot of a subject of FIG. 13A with support columns in another confirmation, overlaid on the diagram.

FIG. 13D shows another diagram of a foot of a subject of FIG. 13A with support columns in another confirmation, overlaid on the diagram.

FIG. 13E shows another diagram of a foot of a subject of FIG. 13A with support columns in another confirmation, overlaid on the diagram.

FIG. 13F shows another diagram of a foot of a subject of FIG. 13A with support columns in another confirmation, overlaid on the diagram.

FIG. 13G shows another diagram of a foot of a subject of FIG. 13A with support columns in another confirmation, overlaid on the diagram.

FIG. 13H shows another diagram of a foot of a subject of FIG. 13A with support columns in another confirmation, overlaid on the diagram.

FIG. 13I shows another diagram of a foot of a subject of FIG. 13A with support columns in another confirmation, overlaid on the diagram.

FIG. 14A shows a top view of an example of another orthopedic insert.

FIG. 14B shows a cross-sectional view of the orthopedic insert of FIG. 14A taken along line 14B-14B of FIG. 14A.

FIG. 15A shows a top view of an example of a base.

FIG. 15B shows a perspective view of another example of a base.

FIG. 15C shows a perspective view of another example of a base.

FIG. 16A shows a perspective view of an interfacing support column having a rectangular protrusion.

FIG. 16B shows a perspective view of the interfacing support column of FIG. 16A having a cylindrical protrusion.

FIG. 16C shows a perspective view of various support columns in coupling arrangement with the base of the orthopedic insert of FIG. 14A.

FIG. 17A shows a partial top view of an example of another interfacing support column.

FIG. 17B shows a cross-sectional view of the interfacing support column of FIG. 17A taken along line 17B-17B of FIG. 17A.

FIG. 18A shows a top view of the orthopedic insert of FIG. 14A installed in an orthopedic boot.

FIG. 18B shows a cross-sectional view of the orthopedic insert of FIG. 14A installed in the orthopedic boot taken along line 18B-18B of FIG. 18A.

FIG. 19A shows a perspective view of another example of an orthopedic insert without the insole and the cushioning layer.

FIG. 19B shows a top view of the orthopedic insert of FIG. 19A without the insole and the cushioning layer.

FIG. 19C shows a cross-sectional view of the orthopedic insert of FIG. 19A with the insole and the cushioning layer taken along line 19C-19C of FIG. 19B.

FIG. 20A shows a top view of another orthopedic insert installed in an orthopedic boot.

FIG. 20B shows a cross-sectional view of the orthopedic insert of FIG. 20A installed in the orthopedic boot, taken along line 20B-20B of FIG. 20A.

FIG. 21A shows a top view of another orthopedic insert installed in an orthopedic boot.

FIG. 21B shows a cross-sectional view of the orthopedic insert of FIG. 21A installed in the orthopedic boot, taken along line 21B-21B of FIG. 21A.

FIG. 22 shows a transverse cross-sectional view of an example of a support column and a wedge.

FIG. 23A shows a front view of another orthopedic insert.

FIG. 23B shows a front view of the orthopedic insert of FIG. 23A, with respective apertures.

FIG. 24A shows a front view of another orthopedic insert.

FIG. 24B shows a mirror image, taken along a vertical axis, of a front view of the orthopedic insert of FIG. 24A.

FIG. 25A shows a top view of another orthopedic insert installed in an orthopedic boot.

FIG. 25B shows a cross-sectional view of the orthopedic insert of FIG. 25A installed in the orthopedic boot taken along line 25B-25B of FIG. 25A.

FIG. 26 shows a front view of the orthopedic insert of FIG. 25A installed in the orthopedic boot.

FIG. 27A shows a top view of another orthopedic insert installed in an orthopedic boot.

FIG. 27B shows a cross-sectional view of the orthopedic insert of FIG. 27A installed in the orthopedic boot taken along line 27B-27B of FIG. 27A.

FIG. 28A shows a perspective view of a biased support column.

FIG. 28B shows a perspective view of another biased support column.

FIG. 29A shows a top view of another orthopedic insert installed in an orthopedic boot.

FIG. 29B shows a cross-sectional view of the orthopedic insert of FIG. 27A installed in the orthopedic boot taken along line 29B-29B of FIG. 29A.

FIG. 30A shows a bottom view of the base of the orthopedic insert of FIG. 29A with a first coupling configuration for a tether.

FIG. 30B shows a bottom view of the base of the orthopedic insert of FIG. 29A with a second coupling configuration for the tether.

FIG. 31 shows a cross-sectional view of the orthopedic insert of FIG. 27A installed in the orthopedic boot taken along line 31-31 of FIG. 29A.

FIG. 32A shows a front view of an orthopedic boot.

FIG. 32B shows a perspective view of the orthopedic boot of FIG. 32A.

FIG. 32C shows a top view of the orthopedic boot of FIG. 32A.

FIG. 33 shows a cross-sectional view of the orthopedic boot of FIG. 32A taken along line 33-33 of FIG. 32C.

FIG. 34 shows a front view of the orthopedic boot of FIG. 32A.

FIG. 35 is an illustration of a bladder of the orthopedic boot of FIG. 32A.

FIG. 36 shows a side view of the orthopedic boot of FIG. 32A with a ring surrounding and encapsulating the support member.

FIG. 37A shows a top view of an example of an orthopedic shoe having an orthopedic insert.

FIG. 37B shows a cross-sectional view of the orthopedic shoe taken along line 37B-37B of FIG. 37A.

FIG. 38 shows another cross-sectional view of the orthopedic shoe taken along line 38-38 of FIG. 37A.

FIG. 39 shows a side view of an example of the orthopedic insert of FIG. 37A.

FIG. 40A shows a side view of a cushioning layer of the orthopedic insert of FIG. 37A.

FIG. 40B shows a side view of an insole with coupled support columns of the orthopedic insert of FIG. 37A.

FIG. 40C shows a perspective view of a base of the orthopedic insert of FIG. 37A interfaced with a sole of the orthopedic shoe of FIG. 37A.

FIG. 40D shows an exploded side view of the orthopedic insert of FIG. 37A, with the sole of the orthopedic shoe of FIG. 37A.

FIG. 41A shows a top view of the cushioning layer of FIG. 40A.

FIG. 41B shows a top view of the insole of FIG. 40B.

FIG. 41C shows a top view of the cushioning layer of FIG. 40A over the insole of FIG. 40B

FIG. 42 shows a side view of the orthopedic insert of FIG. 37A interfacing with the sole of the orthopedic shoe of FIG. 37A.

FIG. 43 shows a side view of the orthopedic insert and the orthopedic shoe of FIG. 37A.

FIG. 44 shows an example of a flowchart of a process for treating or preventing the formation of pressure sores.

FIG. 45A shows a side view of pressure sensor arrays placed on an orthopedic insert of FIG. 2A.

FIG. 45B shows a side view of pressure sensor arrays placed on the orthopedic insert of FIG. 2B with an aperture through the cushioning layer.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

Also, it is to be understood that the use the phraseology and terminology used herein is for the purpose of description and should not be regarded as limiting. Furthermore, the use of “right”, “left”, “front”, “back”, “upper”, “lower”, “above”, “below”, “top”, or “bottom” and variations thereof herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

Unless otherwise specified or limited, phrases similar to “at least one of A, B, and C,” “one or more of A, B, and C,” etc., are meant to indicate A, or B, or C, or any combination of A, B, and/or C, including combinations with multiple or single instances of A, B, and/or C.

In some embodiments, aspects of the present disclosure, including computerized implementations of methods, can be implemented as a system, method, apparatus, or article of manufacture using standard programming or engineering techniques to produce software, firmware, hardware, or any combination thereof to control a processor device, a computer (e.g., a processor device operatively coupled to a memory), or another electronically operated controller to implement aspects detailed herein. Accordingly, for example, embodiments of the invention can be implemented as a set of instructions, tangibly embodied on a non-transitory computer-readable media, such that a processor device can implement the instructions based upon reading the instructions from the computer-readable media. Some embodiments of the invention can include (or utilize) a device such as an automation device, a special purpose or general purpose computer including various computer hardware, software, firmware, and so on, consistent with the discussion below.

The term “article of manufacture” as used herein is intended to encompass a computer program accessible from any computer-readable device, carrier (e.g., non-transitory signals), or media (e.g., non-transitory media). For example, computer-readable media can include but are not limited to magnetic storage devices (e.g., hard disk, floppy disk, magnetic strips, and so on), optical disks (e.g., compact disk (CD), digital versatile disk (DVD), and so on), smart cards, and flash memory devices (e.g., card, stick, and so on). Additionally, it should be appreciated that a carrier wave can be employed to carry computer-readable electronic data such as those used in transmitting and receiving electronic mail or in accessing a network such as the Internet or a local area network (LAN). Those skilled in the art will recognize many modifications may be made to these configurations without departing from the scope or spirit of the claimed subject matter.

Certain operations of methods according to the invention, or of systems executing those methods, may be represented schematically in the FIGS. or otherwise discussed herein. Unless otherwise specified or limited, representation in the FIGS. of particular operations in particular spatial order may not necessarily require those operations to be executed in a particular sequence corresponding to the particular spatial order. Correspondingly, certain operations represented in the FIGS., or otherwise disclosed herein, can be executed in different orders than are expressly illustrated or described, as appropriate for particular embodiments of the invention. Further, in some embodiments, certain operations can be executed in parallel, including by dedicated parallel processing devices, or separate computing devices configured to interoperate as part of a large system.

As used herein in the context of computer implementation, unless otherwise specified or limited, the terms “component,” “system,” “module,” etc. are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

As used herein, the term, “controller” and “processor” and “computer” include any device capable of executing a computer program, or any device that includes logic gates configured to execute the described functionality. For example, this may include a processor, a microcontroller, a field-programmable gate array, a programmable logic controller, etc.

The following discussion is presented for a person skilled in the art to make and use embodiments of the invention. Various modifications to the illustrated embodiments will be readily apparent to those skilled in the art, and the generic principles herein can be applied to other embodiments and applications without departing from embodiments of the invention. Thus, embodiments of the invention are not intended to be limited to embodiments shown, but are to be accorded the widest scope consistent with the principles and features disclosed herein. The following detailed description is to be read with reference to the figures, in which like elements in different figures have like reference numerals. The figures, which are not necessarily to scale, depict selected embodiments and are not intended to limit the scope of embodiments of the invention. Skilled artisans will recognize the examples provided herein have many useful alternatives and fall within the scope of embodiments of the invention.

As detailed above, pressure sores (and ulcers) impose a significant burden to the healthcare field, which manifests itself into many different forms. For example, the formation of pressure sores can result in complications, such as infections, swelling, and bleeding. Not only do these complications introduce unwanted risks for the patient (and others exposed to the complication such as an infection), but they also sap significant time, attention, and other resources from the healthcare professionals (e.g., physicians, nurses, etc.). Aside from the issues imposed on the healthcare system, pressure sores can be painful, uncomfortable, and can increase the time required for full recovery (e.g., following an orthopedic surgery).

Addressing issues related to pressure sores requires overcoming at least two main challenges. First, how can pressure ulcers be prevented (or the risk significantly decreased), and second how can a pressure sore be treated properly following its formation. Generally, following an orthopedic surgery the appendage must be fixed (or its range of motion significantly inhibited) to facilitate proper internal healing (e.g., of bones). However, typical orthopedic products, such as off the shelf boots and traditional casts, are significantly flawed—encouraging ulcer formation and inhibiting healing of previously formed ulcers. These typical orthopedic products are unable to offload pressure generally, and are unable to selectively offload pressure to particular areas (such as locations where sores are more likely to develop). Rather, typical orthopedic products are formed of contiguous planar inserts (for shoes or boots) that do not remove or significantly mitigate pressure from locations on the patient's foot (e.g., such as when the patient's body weight is subjected on the insert). In other words, a pressure profile spans across the entire foot of the subject including regions that include or are prone to pressure sore formation.

Some conventional approaches have attempted to prevent or treat ulcers, but these approaches do not sufficiently address the underlying problem, and rather introduce other undesirable problems. In one conventional approach, a concavity (or aperture) was introduced into the supporting structure (or pad insert) of the orthopedic boot. However, the anatomy between individuals is highly variable, and pressure sores do not always form in the same location or region. Thus, regions that are more prone to pressure sore formation may not be disposed near the concavity for some individuals, and regardless, pressure sores are still free to develop elsewhere. Other conventional approaches propose the manipulation of orthopedic pad inserts, where some hexagonal pad inserts can be cut out. However, these hexagonal pad inserts are designed poorly. For example, while these hexagonal pad inserts may have a cutout, these do not completely or effectively offload pressure at the location of the cutout. In other words, the hexagonal pad inserts are not structurally sound to prevent contact between the pressure sore (or region prone to forming pressure sores) and the orthopedic boot. In fact, generally, the hexagonal pad inserts collapse and lose integrity with regular use. Additionally, these hexagonal pads cannot be put back together after the hole has been formed.

Some embodiments of the disclosure provide improvements over the conventional systems described above, and others, for preventing and managing sores, ulcers, and wounds. For example, some systems and methods according to embodiments of the disclosure can selectively support, or remove contact between portions of an orthopedic boot (or shoe) and portions of the patient's foot. The ability to selectively adjust how the patient's foot is supported (or not supported, such as being free of contact) is desirable for at least a few reasons. First, a user (or other practitioner) can, in the case of preventing pressure sores, avoid supporting regions, and thus contacting regions of the foot (with the orthopedic boot) that are prone to developing pressure sores. Second, a user can, in the case of treating (or managing) already existing pressure sores, can offload the pressure sore, thus freeing the contact between the pressure sore and the orthopedic boot. In either case (e.g., the prevention or treatment of pressure sores) the selective loading and offloading of particular regions of the patient's foot can be advantageous to promote healing of already formed sores, or mitigating risks of forming sores.

In some embodiments, an orthopedic insert (or orthopedic boot) can address previous problems by utilizing removable support columns. The support columns are removably coupled to various locations on a base (or sole) that interfaces with the sole of the orthopedic boot (or shoe). The support columns are located between the sole of the shoe and a insole (e.g., a rigid plate) that a foot of a subject rests on. A void can be formed in a particular region on the insole (e.g., such as a location of an ulcer, or a location prone to the formation of ulcers). This way, and depending on the location of the support columns, the void surrounds a location of an ulcer (or other region), thus preventing contact (or offloading pressure) between a portion of the orthopedic insert (or boot) and the ulcer (or other region). As such, the ability to manipulate the location of the support columns allows for adjusting the loading of various regions on the patient's foot, which can address the specific needs of a patient. In some embodiments, the support columns can be adjusted (e.g., a cross-section, a height, material properties, etc.) so as to adjust the loading at the particular column. For example, the cross-section of a column can be increased (or by the addition of additional adjacent support columns), which can increase the loading by the column (from the weight of the patient) thereby adjusting the pressure profile (or loading profile) of the orthopedic insert.

In some embodiments, the orthopedic insert includes at least two different types of support columns. One type of support column resembles a screw-in column, which includes a fastener that couples together the screw-in column to the insole, providing structural integrity in the particular location. The second type of support column is a lock-in column, which includes a fattener that couples the lock-in column to the base, distributing the loading from the weight of the patient, and more particularly distributing the loading such that the loading at the screw-in column can be adjusted (e.g., decreased).

In some embodiments, a support member of an orthopedic boot or shoe (e.g., the structural shell) can include an expandable shell (e.g., a bump out section) surrounding the heel of a patient. In this case, the expandable shell can have one or more sections that expand (e.g., “bump out”) or retract away or towards the heel of the patient, as desired by a user. Typical orthopedic boots completely surround the entire foot and cannot expand when the orthopedic boot is secured to the patient, such as when Velcro® fastener straps are tightened. This inability to selectively expand a region of the orthopedic boot can cause irritation to areas on the heel, sides, and top of the foot (or ankle), and especially so for cases of wounds (incisions) or swelling. The expandable shell (or “bump out” capability) according to some embodiments, prevents or mitigates the possibility of contact between a portion of the orthopedic boot and an undesirable portion (such as a wound), which facilitates healing of the wound.

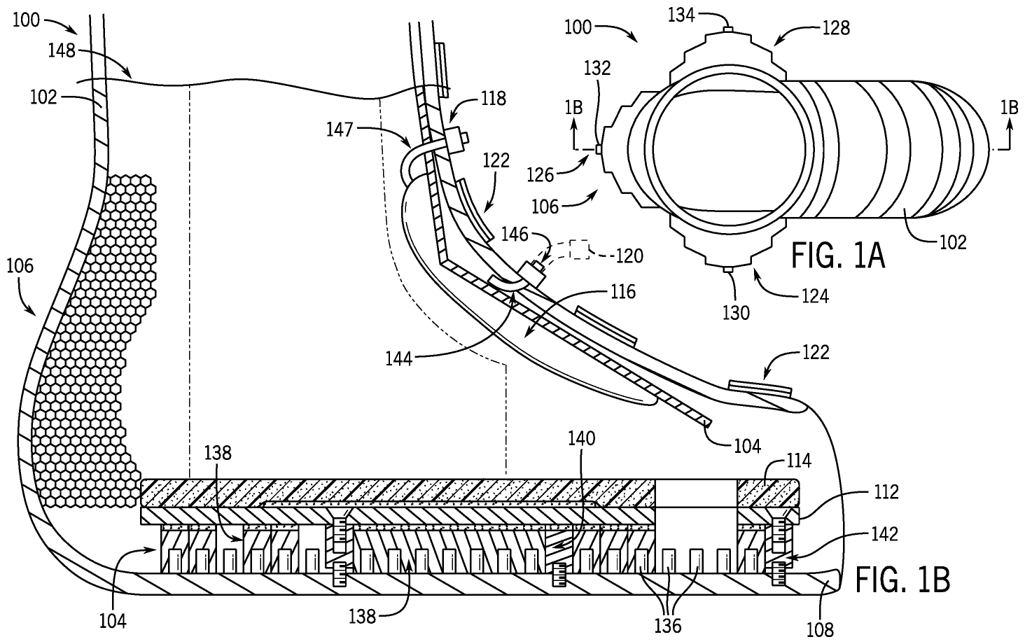

FIGS. 1A and 1B show an example of an orthopedic boot 100, according to some embodiments of the disclosure. The orthopedic boot 100 includes a support member 102, support columns 104, an expandable shell 106, a sole 108, support columns 110, an insole 112, a cushioning layer 114, a bladder 116, a deflate button 118, a pump 120, and fastening assemblies 122. As shown in FIGS. 1A and 1B, the support member 102 is structured as having a frame and a lining, which surrounds and secures a foot of the subject within the orthopedic boot 100. In some cases, and as shown, supporting plates 104 are coupled (or connected) to the frame and lining of the support member 102, providing structural rigidity to the orthopedic boot 100. The supporting plates 104 can be formed of many different materials (e.g., plastics including high density polyethylene, metals, etc.) and can embody many different shapes. In some cases, some, such as two of the supporting plates 104, can be generally L-shaped and positioned on opposing lateral sides of the support member 102 (e.g., on a medial side and a lateral side). Other supporting plates 104 can be planar and positioned on a front side (e.g., over a shin), a rear side (e.g., over the Achilles tendon), and a top side (e.g., over a top portion of a foot) of the support member 102. In the illustrated embodiment of FIGS. 1A and 1B, an L-shaped supporting plate 104 is coupled to the support member 102 such that the L-shaped supporting plate 104 is adjacent to a top of a foot and a shin of the subject.

The expandable shell 106 is connected to and interfaces with the support member 102. In some embodiments, a periphery of the expandable shell 106 is connected to (e.g., by fasteners, stitches, adhesive, etc.) an aperture formed in the support member 102 to create an interface between the support member 102 and the expandable shell 106. In some cases, the expandable shell 106 has a single expandable domain, whereas in other cases, such as the illustrated embodiment of FIGS. 1A and 1B, the expandable shell 106 has expandable domains 124, 126, 128. The expandable domains 124, 126, 128 have corresponding tabs 130, 132, 134. The tabs 130, 132, 134 are coupled to, connected to, or integrally formed with the corresponding expandable domain. The expandable domains 124, 126, 128 are strategically situated so as to envelop a particular region of the foot of the subject prone to pressure sore formation. For example, the expandable domain 124 can be configured to surround the lateral malleolus of the subject, the expandable domain 126 can be configured to surround the heel of the subject (e.g., a portion of the calcaneus), and the expandable domain 128 can be configured to surround the medial malleolus of the subject.

In some embodiments, the expandable shell 106 (including the expandable domains 124, 126, 128) can be selectively expanded or retracted to increase or decrease the internal volume defined within a given expandable domain (or the entire expandable shell). This way, the expandable shell 106 can adjust how the expandable shell 106 contacts the foot of the subject (e.g., completely removing contact between the expandable shell or expandable domain and the foot of the subject). In some embodiments, the tabs 130, 132, 134 allow a user to more easily grasp and pull or compress the expandable domains 124, 126, 128. As shown in FIGS. 1A and 1B, the expandable shell 106 is formed of a tessellated hexagonal expandable material (e.g., a rubber material). However, in other embodiments other tessellated shapes (or combinations of shapes) can be used for the expandable shell 106 such as, squares, triangles, etc.

The sole 108 has a bottom surface that engages with a walking surface, and has an opposite upper surface. The sole 108 can be structured as known in the art, such as having a heel, and as being generally rigid. The sole 108 can be formed of various materials, such as plastics, metals, polymers, rubbers, etc. As illustrated, the sole 108 includes a plurality of pegs 136 upwardly extending from the upper surface. The pegs 136 can be connected to the sole 108 in many different ways, such as using an adhesive, fasteners, etc. In some configurations, the pegs 136 are integrally formed with the sole 108. The pegs 136 are cylindrical in shape, however, in other embodiments, the pegs can embody different shapes such as, for example, prisms (e.g., triangular prisms, rectangular prisms, octagonal prisms, combinations thereof, etc.), or any three-dimensional geometric shape (e.g., conical or frustoconical). The pegs 136 are rigid and can be formed of various materials (e.g., plastics, metals, polymers, rubbers, etc.).

As shown, the pegs 136 are configured to receive and retain at least some of the support columns 110. More specifically, the support columns 110 include nesting support columns 138. Each of the nesting support columns 138 include an inwardly directed bore (e.g., directed through a base surface of the nesting support column 138) that corresponds in shape with the shape of the pegs 136. For example, in the illustrated embodiment, the pegs 136 are generally cylindrical, and thus the inwardly directed bore is cylindrical. However, in alternative embodiments, such as when the pegs 136 are shaped as other prisms, the inwardly directed bore corresponds to the shape of the pegs 136. As shown, a peg 136 is inserted into the inwardly directed bore of given nesting support column 138 to secure the given nesting support columns 138 to the peg 136 and thus to the sole 108. In some configurations, a given peg 136 can be implemented as a bolt body, having threading. In this configuration, the inwardly directed bore of the nesting support column 138 can also include threading, such that the bolt body threadingly engages the inwardly directed bore of the nesting support column 138 to secure the nesting support column 138 to the bolt body and thus to the sole 108.

The support columns 110 also include fastening support columns 140, and locking support columns 142. The fastening support columns 140 include a downwardly extending bolt body that includes threads. The fastening support column 140 can be rotated to threadingly engage a threaded bore directed into the sole 108, to secure the fastening support column 140 to the sole 108. The locking support columns 142 also include a downwardly extending bolt body that is structured and functions similarly to the fastening support columns 140. However, the locking support columns 142 also include a fastener (e.g., a screw, a bolt, etc.) that is directed through the insole 112 and an upper surface of the locking support column 142 to secure the insole 112 to the locking support column 142. In some embodiments, the support columns 110 can be secured to the insole 112 using fastening systems and methods used in the art (e.g., adhesives, etc.). In the illustrated embodiment, the support columns 110 are secured to the insole 112 by a hook and loop fastener (e.g., Velcro® fastener).

In some embodiments, the support columns 110 include a body that defines (or partial defines) the structural integrity for the given support column 110. For example, as described above, the body can embody various shapes (e.g., prisms, cylinders, etc.) and thus the structural integrity of the support column 110 differs between differing embodiments. In some cases, the cross-sectional area of the body of the support columns 110 can differ between other support columns 110 (e.g., increased, decreased, change in shape, etc.). In some embodiments, the height of the body of the support columns 110 can differ between other support columns 110 (e.g., increased, decreased, change in shape, etc.). In some embodiments, the structural rigidity of body of the support columns 110 can differ between other support columns 110 (e.g., increased, decreased, etc.). For example, the material composition of the body of the support columns 110 can differ between other support columns 110, which may impact the deformability of the support columns 110. As a more specific non-limiting example, the body of one support column 110 may be formed of a rigid material (e.g., a plastic, a metal, etc.), whereas the body of another support column 110 may be formed of a deformable material (e.g., a foam, a polymer, etc.) that may have a desirable elasticity (e.g., nearly returning to its desired shape after unloading).

As shown, the insole 112 is connected to the support columns 110 and is situated above the pegs 136 and the sole 108. The insole 112 can be shaped as desired, such as contouring the geometry of the foot of the subject. In some cases, and as illustrated, the insole 112 can include planar portions so as to interface with the support columns. In some configurations where the insole 112 has a curvature, a top surface of the support columns 110 can have a similar curvature or profile as the insole 112 to generate a better interface between the support columns 110 and the insole 112. The insole 112 can be structured to have a desired rigidity. For example, in some cases the insole 112 is rigid to sufficiently support a foot of a subject, however, in some cases the insole 112 may deform (e.g., slightly after unloading).

In the illustrated embodiment of FIGS. 1A and 1B, a cushioning layer 114 is connected to and situated above the insole 112. The cushioning layer 114 generally provides comfort to the foot of the user, when secured within the support member 102 of the orthopedic boot 100. For example, the cushioning layer 114 is deformable such that the cushioning layer 114 contours the foot of the user when the food is provides a loading force to the cushioning layer 114 (e.g., from the weight of the subject). In some configurations, the cushioning layer 114 can be formed of a material that includes a memory foam (e.g., polyurethane).

The bladder 116 is shown connected to the support member 102 (e.g., by adhesive, fasteners, etc.) and is situated within the interior volume of the support member 102. As shown, the bladder 116 lines a jutting edge of an L-shaped support plate 104. This way, when the bladder 116 inflates, a foot positioned within the orthopedic boot 100 contacts (and is cushioned by) the bladder 116 rather than the jutting edge of the L-shaped support plate 104. The bladder 116 includes a conduit 144 in fluid communication with the interior of the bladder 116, and coupled to the support member 102 (e.g., via a valve 146). The valve 146 can be configured to receive a pump 120 to provide a source of fluid (e.g., air) to the bladder 116 thereby inflating the bladder 116. In some configurations, the valve 146 can be structured as a one-way valve to prevent fluid from escaping out of the bladder 116, while allowing fluid to enter the bladder 116 (e.g., from the pump 120). Another conduit 147 can be connected to the bladder 116 and provide fluid communication between the bladder 116 and a deflate button 118 (e.g., configured as a one-way valve). The deflate button 118, when depressed, allows for fluid communication between the bladder 116 and the ambient environment. In other words, if the bladder 116 is inflated, and the deflate button is depressed, fluid will flow from the bladder 116 and into the ambient environment.

In some embodiments, the orthopedic boot 100 can include a number of fastening assemblies 122 that secure the support member 102 to the foot or leg of the subject. In some cases, the fastening assemblies 122 can include hook and loop fastening straps, and loops, such that a strap is directed through the loop and fastened to itself to adjust the tension on the strap. In other embodiments, the fastening assemblies 122 can be structured in different ways.

In some embodiments, although the orthopedic boot 100 is described as being a boot (e.g., where the support member 102 is secured to the leg of the subject), in other embodiments the orthopedic boot 100 can be cut along 148 to create an orthopedic shoe. In this case, the support member 102 (and corresponding components such as the support plates 104) do not contact a leg of a subject. Additionally, in some embodiments, the sole 108 can be replaced with a base, as described below, such that the support columns 104 are removably coupled to the base (e.g., the base including the pegs 136). In this configuration, the base, which forms part of an orthopedic insert can interface with the sole 108.

As previously described, the sole 108 includes the pegs 136 and threaded bores to removably couple the support columns 110 to the sole 108. In other embodiments, and as discussed in more detail below, a base can be interchanged with the sole 108, such that the base has the pegs 136 and threaded bores. This way, the base and other corresponding components define an orthopedic insert 200, that can be secured within the orthopedic boot 100, other boots, and other shoes.

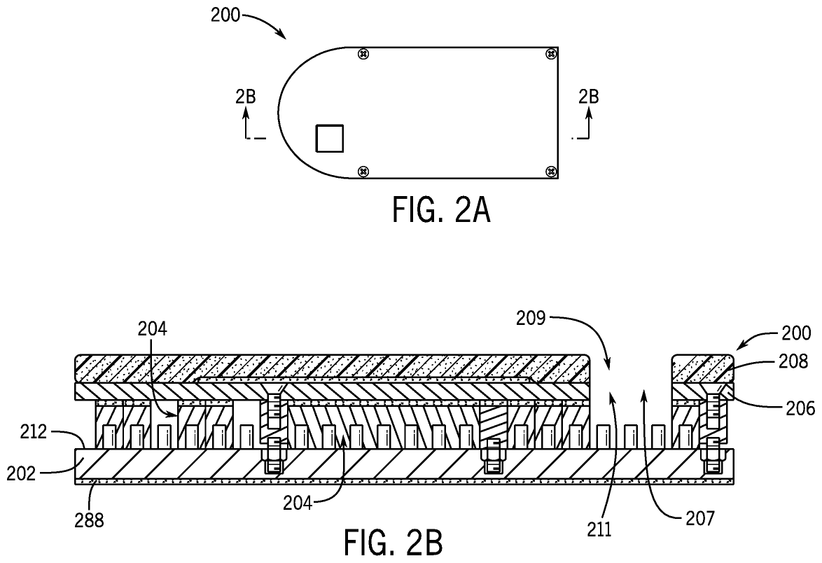

FIGS. 2A and 2B show an example of an orthopedic insert 200 according to some embodiments of the disclosure. The orthopedic insert 200 includes many of the previously discussed components with regard to the orthopedic boot 100. Thus, the previously discussed components of the orthopedic boot 100 also pertain to the orthopedic insert 200. The orthopedic insert 200 includes a base 202, support columns 204, an insole 206, and a cushioning layer 208. As described above, the components with similar names but differing reference numerals, such as the support columns 110 and the support columns 204, are structured and function similar to each other. Additionally, the descriptions for each component pertain to each other. As shown, the base 202 includes a lower surface 210, an opposite upper surface 212, and pegs 214 upwardly extending from the upper surface 212 (e.g., being connected, coupled, integrally formed, etc.). The support columns 204 are removably coupled to the corresponding pegs 214, and are shown as being in a coupled configuration. In some embodiments, the pegs 214 can all be the same shape and size.

In some embodiments, and as illustrated, the base 202 includes bores 216 directed into the upper surface 212 of the base 202. Each bore 216 can correspond to the shape of structures of the support columns 204 (e.g., a bolt body of a support column 204). In a more specific non-limiting example, the bores 216 can be cylindrical and can include threads to threadingly engage a given support column 204. The base 202 can be structured to have a desired rigidity. For example, in some cases, the base 202 can be rigid, and can be formed of corresponding materials (e.g., plastics including high density polyethylene, metals, etc.). In some embodiments, the insole 206 is connected to the support columns 204 (e.g., via fasteners, adhesives, etc.) and is situated above the pegs 214 and the base 202. The insole 206 can be shaped as desired, such as contouring the geometry of the foot of the subject. In some cases, and as illustrated, the insole 206 can include planar portions so as to interface with the support columns. The cushioning layer 208 is connected to the insole 206 and is situated above the base 202, the support columns 204, and the insole 206. The cushioning layer 208 generally provides comfort to the foot of the user. For example, the cushioning layer 208 is deformable such that the cushioning layer 208 contours the foot of the user when the foot provides a loading force to the cushioning layer 208 (e.g., from the weight of the subject). In some configurations, the cushioning layer 208 can be formed of a material that includes a memory foam (e.g., polyurethane).

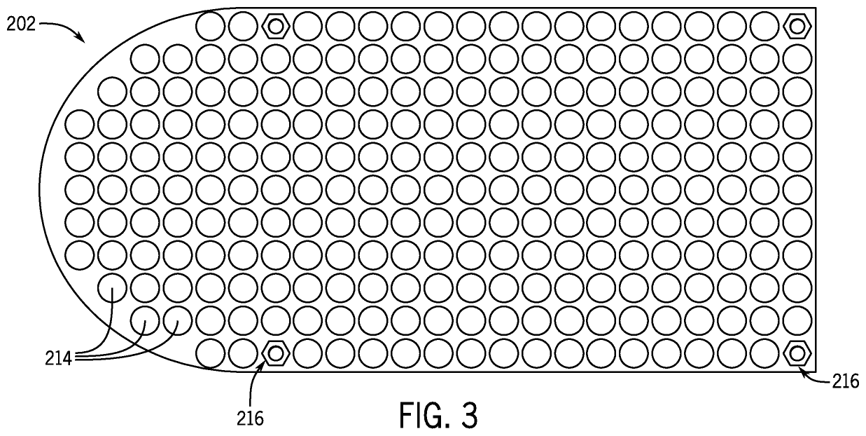

FIG. 3 shows an example of the base 202 of the orthopedic insert 200 having upwardly projecting pegs 214. The base 202 is illustrated as having a simplified shape, however, in alternative configurations, the base 202 can be shaped to correspond to any number or types of footwear (e.g., boots, shoes, sandals, etc.). This way, the orthopedic insert 200, including the base 202, can be securely received within the desired type and size (e.g., standardized sizes, such as for men and women) of footwear. The pegs 214 are arranged as an array, such that the spacing between adjacent pegs 214 is uniform across the entire base 202. However, in alternative embodiments, the pegs 214 can be spaced apart at non-uniform intervals. For example, regions of the base 202, such as centrally located regions of the base 202, may have pegs 214 that are spaced apart at greater distances than peripheral regions of the base 202. The pegs 214 can be rigid and can be formed of various materials (e.g., plastics, metals, polymers, rubbers, etc.). In some embodiments, any of the pegs 214 can be substituted for bores 216. For example, as shown, a number of locations on the base 202 have bores 216 rather than pegs 214. The bores 216 are shown as being situated near the periphery of the base 202, however, in other configurations, other locations can be used for the bores 216.

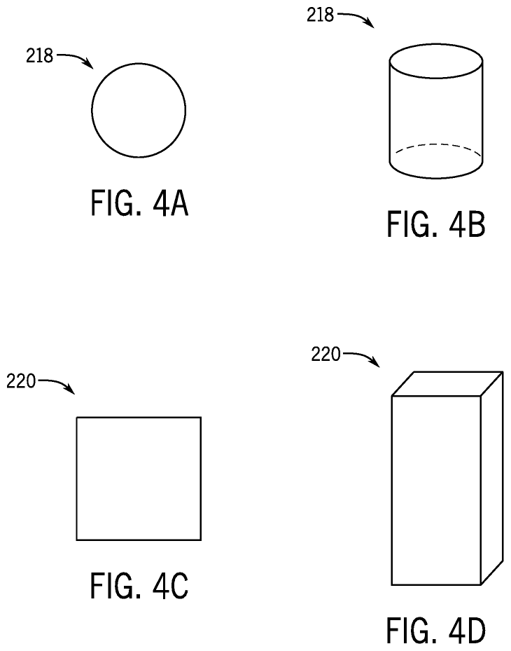

FIGS. 4A-4D show examples of different embodiments of the pegs 214. Specifically, FIG. 4A shows an example of a top view and FIG. 4B shows a perspective view of a cylindrical peg 218, which is a specific non-limiting implementation of the pegs 214. FIG. 4C shows an example of a top view and FIG. 4D shows an example of a perspective view of a rectangular peg 220, which is a specific non-limiting implementation of the pegs 214. The pegs 218 are cylindrical, while the pegs 220 are rectangular, however, in other embodiments, the pegs can embody different shapes such as, for example, other prisms (e.g., triangular prisms, rectangular prisms, octagonal prisms, combinations thereof, etc.).

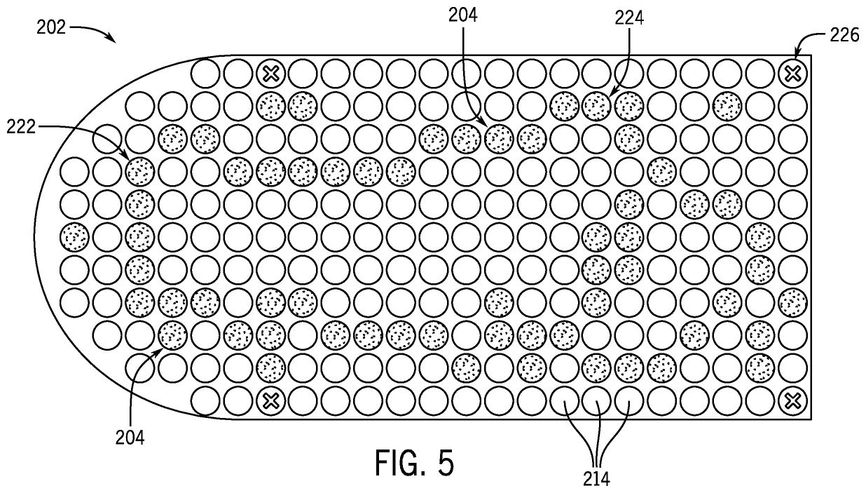

FIG. 5 shows an example of support columns 204 coupled to the base 202. As described above, the support columns 204 include many different types. For example, the support columns 204 can be implemented as a nesting support column 222, a fastening support column 224, and a locking support column 226. The previous description of the nesting support column 138, fastening support column 140, and the locking support column 142, pertains to the discussion of the nesting support columns 222, the fastening support columns 224, and the locking support columns 226, and vice versa. As shown, the locking support columns 226, which can provide additional structural support, as positioned around the periphery of the base 202. Conversely, the nesting support columns 222 and the fastening support columns 224 are positioned near the interior (or central region) of the base 202.

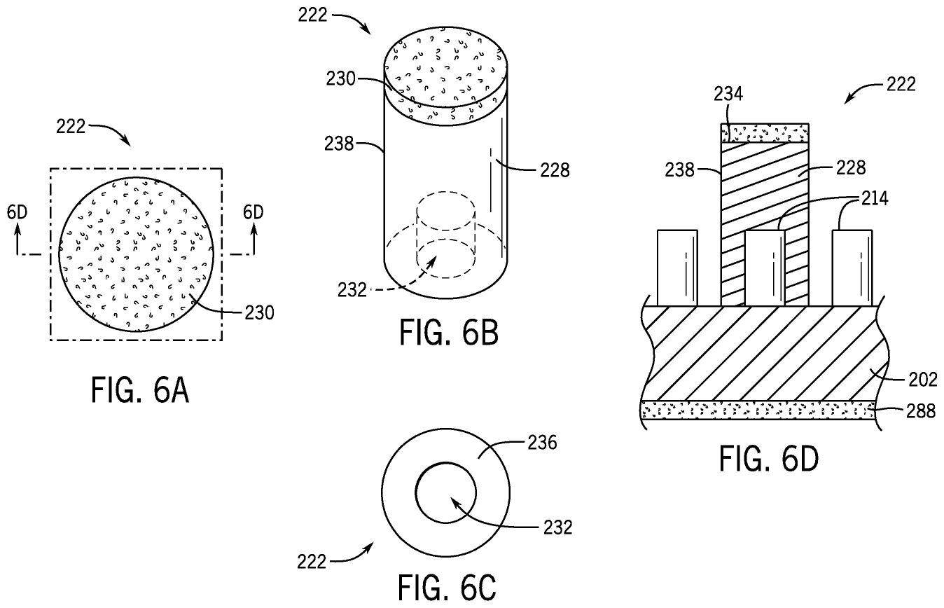

FIGS. 6A-6D show an example of a nesting support column 222. The nesting support column 222 includes a body 228, a coupling layer 230, and a recess 232. The body 228 is illustrated as being cylindrical, however, in alternative embodiments the body 228 can embody different shapes (or profiles) which can be, for example, prisms (e.g., triangular prisms, rectangular prisms, octagonal prisms, combinations thereof, etc.), or any three-dimensional geometric shape (e.g., conical, frustoconical) an I-beam, etc.). The body 228 can be formed of a rigid material (e.g., a plastic, a metal, etc.) to provide structural integrity to the orthopedic insert 200. In other embodiments, however, the body 228 can be slightly deformable (e.g., being formed of orthotic materials such as foams, cork, Plastazote® polyethylene foam). The body 228 includes an upper surface 234, a lower surface 236, and an adjacent surface (or surfaces) 238 defined between the upper surface 234 and the lower surface 236.

In some embodiments, the coupling layer 230 is connected (or joined) to the upper surface 234 of the body 228 and is configured to removably couple (or connect) the nesting support column 222 to the insole 206. In one implementation, the coupling layer 230 can include an adhesive layer situated underneath a film backing. This way, when the nesting support column 222 is coupled to the base 202, the film backing can be removed and the coupling layer 230 can be adhered to the insole 206. In another implementation, the coupling layer 230 can include a hook fastener or a loop fastener (e.g., Velcro® fastener), and a bottom surface of the insole 206 can include the other of the hook fastener or the loop fastener. This way, the coupling layer 230 of the nesting support column 222 can be removably coupled to the insole 206.

FIG. 6D specifically shows the nesting support column 222 interfacing with a peg 214 to couple the nesting support column 222 to the base 202. The peg 214 is inserted into the recess 232 of the nesting support column 222 until the pegs contacts an upper surface of the recess 232, as shown. In some embodiments, the shape of the recess 232 corresponds with the shape of the pegs 214, where the recess 232 is illustrated as being cylindrical. In other configurations, as described above, the recess 232 can be shaped differently. In some implementations, a gap may be formed between the upper surface of the recess 232 and a portion of the peg 214. In other words, the peg 214 may not extend into the entirety of the recess 232. The adjacent surface 238 contacts adjacent pegs 214, when the nesting support column 222 is coupled to the given peg 214. In other configurations, such as those in which the nesting support columns 222 (or the pegs 214) are not cylindrically shaped (e.g., a rectangular prism) the body 228 of the nesting support column 222 may have additional adjacent surfaces 238 where respective adjacent surfaces 238 contact respective adjacent pegs 214. In some embodiments, the adjacent surface 238 of the body 228 need not contact the adjacent pegs 214. For example, a tolerance between the adjacent surface 238 and the adjacent pegs 214 may exist. In some embodiments, the peg 214 or the body 228 (such as within the recess 232) can include a locking feature, such that when the nesting support column 222 rotates, the locking feature is engaged to prevent axial movement of the nesting support column 222.

In some embodiments, the recess 232 and the upper surface 212 of the base can include a coupling layer. In some configurations, the recess 232 can be magnetically coupled to the base 202. For example, in some cases, the coupling layer of the recess 232 can include a magnet, and the upper surface 212 of the base 202 can include another magnet (e.g., embedded within the base 202, or joined to the surface). This way, when the magnet of the coupling layer of the nesting support column 222 is brought into contact with the another magnet of the upper surface 212 of the base 202, the nesting support column 222 is attracted and coupled to the base 202. This way, the nesting support column 222 does not (and cannot axially move) relative to the base 202, when coupled to the base 202. In other configurations, the coupling layer of the recess 232 can include one of a hook fastener or a loop fastener, and the upper surface 212 of the base 202 can include the other of the hook fastener or the loop fastener, such that when the nesting support column 222 is coupled to the base 202, the nesting support column 222 does not (and cannot) axially move relative to the base 202. Other removably coupling fastening configurations could also be used. Additionally, as described below, the protrusion (or body) of the nesting support column 422 can also have a coupling layer (e.g., a magnet, hook and loop fastener, an adhesive, etc.).

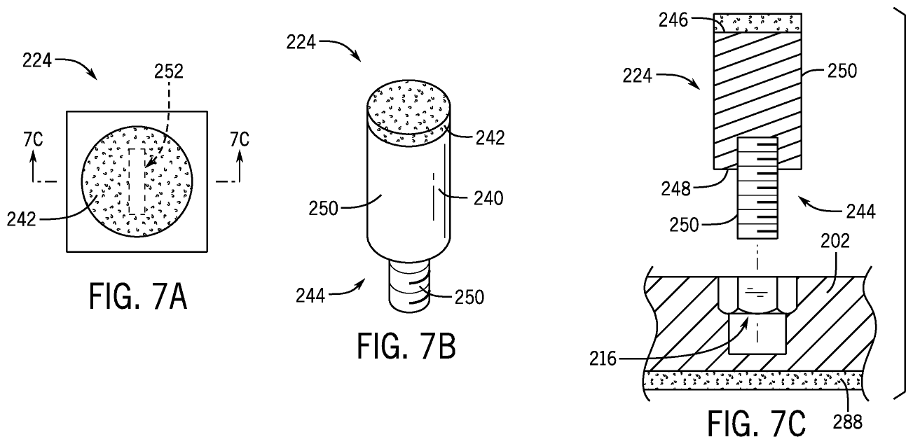

FIGS. 7A-7C show an example of a fastening support column 224. The fastening support column 224 includes a body 240, a coupling layer 242, and a fastener 244. The body 240 is similar to the body 228 of the nesting support column 222, and thus the description of the body 228 pertains to the body 240. The body 240 includes an upper surface 246, a lower surface 248, and an adjacent surface (or surfaces, such as in the case of support columns with different shapes) 250 defined between the upper surface 246 and the lower surface 248. The coupling layer 242 is connected (or joined) to the upper surface 246 of the body 240 and is configured to removably couple (or connect) the fastening support column 224 to the insole 206. The coupling layer 242 is similar to the coupling layer 230 of the nesting support column 222, and thus the description of the coupling layer 230 pertains to the coupling layer 242. In some configurations, the coupling layer 242 can include a tool recess 252 (or a tool protrusion) inwardly (or outwardly) directed into the surface of the coupling layer 242 (e.g., the free end of the coupling layer 242). This way, as will be described in more detail below, the tool recess 252 receives a tool (e.g., a screwdriver, etc.) to transfer torque from the tool and into the fastening support column 224 thereby rotating the fastening support column 224. In some configurations, the tool recess 252 can be a slot drive, a hex socket drive, a cross drive, a Philips head drive, etc.

In some embodiments, and as illustrated, the fastener 244 is implemented as a bolt body 250 extending through the lower surface 248 of the fastening support column 224. The bolt body 250 includes threads that are configured to threadingly engage the threaded bore 216 of the base 202, can be formed of various rigid materials (e.g., metals, plastics, etc.), and can be connected to the bolt body 250 in various ways (e.g., adhesives, integrally formed, etc.). Thus, the fastening support column 224 is removably coupled to the base 202 (e.g., via the threaded bore 216). To install the fastening support column 224, a desired threaded bore 216 is chosen. Then, the bolt body 250 is interfaced with the threaded bore 216, and the fastening support column 224 is rotated in a first direction to advance the bolt body 250 into the threaded bore 216. As described above, a tool can be received within the tool recess 252 to more easily rotate the fastening support column 224. In some configurations, the entirety of the bolt body 250 can be treated, whereas in other configurations the entire bolt body 250 does not include threads so as to allow for a non-threaded surface of the bolt body 250. Once the fastening support column 224 is secured to the base 202, the a portion of the insole 206 can be removably coupled (or coupled) to the fastening support column 224 (e.g., via the coupling layer 242). In some embodiments, as described above with regard to the orthopedic boot 100, the bolt body 250 of the fastening support column 224 can be replaced with an inwardly directed threaded bore 216, and vice versa (e.g., the threaded bore 216 replaced with a bolt body 250). For example, the bolt body 250 extends upwardly from the base 202, and in some cases, can be formed from a peg 214. In this configuration, the bolt body 250 of the base 202 is received within the threaded bore 216 of the fastening support column 224, and the fastening support column 224 is rotated to couple the fastening support column 224 to the base 202.

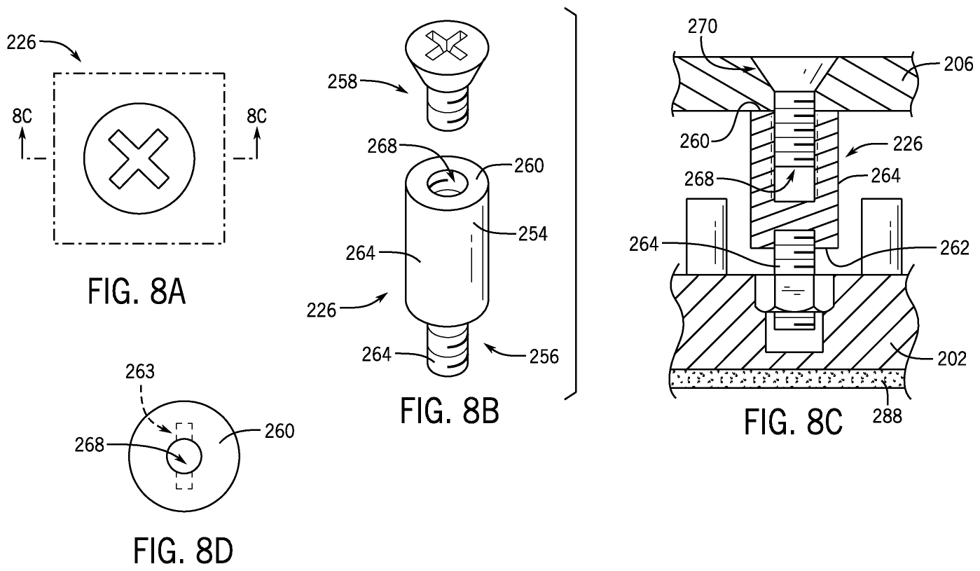

FIGS. 8A-8D show an example of a locking support column 226. The locking support column 226 includes a body 254, a first fastener 256, and a second fastener 258. The body 254 is similar to the body 228 of the nesting support column 222, and thus the description of the body 228 pertains to the body 254. The body 254 includes an upper surface 260, a lower surface 262, and an adjacent surface (or surfaces, such as in the case of support columns with different shapes) 264 defined between the upper surface 260 and the lower surface 262. Similarly to the fastening support column 224, the first fastener 256 is implemented as a bolt body 264 extending through the lower surface 262 of the locking support column 226. The bolt body 264 includes threads that are configured to threadingly engage the threaded bore 216 of the base 202, and can be formed of various rigid materials (e.g., metals, plastics, etc.), and can be connected to the bolt body 264 in various ways (e.g., adhesives, integrally formed, etc.). Thus, the locking support column 226 can be removably coupled to the base 202 (e.g., via the threaded bore 216). The opposing upper surface 260 of the locking support column 226 (or body 254) includes an inwardly directed threaded bore 268, which is configured to receive (and secure) the second fastener 258 to the locking support column 226. In some cases, the upper surface 260 of the support column 226 also includes a tool recess 263 (or protrusion) inwardly (or outwardly) directed into the upper surface 260. The tool recess 263 (or protrusion) is configured to a tool (e.g., a screwdriver, etc.) to transfer torque from the tool and into the locking support column 226 thereby rotating the locking support column 226. FIG. 8D shows one implementation of a tool recess 263 that overlaps at least a portion of the threaded bore 268. Although the tool recess 263 is shown in FIG. 8D as being a slot drive (e.g., for use with a flat head screwdriver), in other configurations other drives can be used. Additionally, the portion of the tool recess 263 that does not overlap with the threaded bore 268 does not need to extend the entire axial length of the threaded bore 268. In some cases, the axial length of the tool recess 263 is less than the axial length of the threaded bore 268.

In some embodiments, the second fastener 258 can be implemented as various fasteners typically used in the art (e.g., bolts, screws, etc.). In some configurations, the second fastener 258 can be implemented as a screw, such as in the illustrated embodiment. In some embodiments, the second fastener 258 is configured to be received through the insole 206 and into the threaded bore 268 to couple the insole 206 to the locking support column 226. Some aspects of the installation of the locking support column 226 are similar to the installation of the fastening support column 224. For example, the bolt body 264 is received within and threadingly engages with the threaded bore 216 to couple the locking support column 224 to the base 202. In some configurations, a tool can be received within the tool recess 263 to more easily rotate the locking support column 226. Once the locking support column 226 is secured to the base 202, the second fastener 258 can be removed (e.g., by rotation), if the second fastener 258 is engaged with the locking support column 226. Then, the insole 206 is positioned such that the insole 206 contacts the upper surface 260 of the locking support column 226, and an aperture 270 directed into and through the entirety of the insole 206 overlaps with the threaded bore 268. The aperture 270 can be sized so as to receive the body of the second fastener 258, and block the receiving of the head of the second fastener 258. Additionally, the aperture 270 can be shaped to contour the head of the second fastener 258, such as illustrated in FIG. 8C. Once the aperture 270 and the threaded bore 268 are sufficiently aligned, the second fastener 258 threadingly engages and is rotated in a first direction to couple the locking support column 226 to the insole 206 with the second fastener 258. This way, the second fastener 258 directly joins together the insole 206 and the locking support column 226, which can prevent tilting or rotation of the insole 206 (e.g., when a foot is received on the insole 206). In some configurations, the second fastener 258 may have a pointed end, and the threaded bore 268 (and the aperture 270) can be removed, such that by rotating the fastener 258 the threaded fastener is advanced through the insole 206 and the body 254 to couple the insole 206 to the locking support column 226.

In some embodiments, and similarly to the description above with regard to the fastening support column 224 (and the orthopedic boot 100), the bolt body 264 of the locking support column 226 can be replaced with an inwardly directed threaded bore 216, and vice versa (e.g., the threaded bore 216 replaced with a bolt body 264). For example, the bolt body 264 extends upwardly from the base 202, and in some cases, can be formed from a peg 214. In this configuration, the bolt body 264 of the base 202 is received within the threaded bore 216 of the locking support column 226, and the locking support column 226 is rotated to couple the locking support column 226 to the base 202.

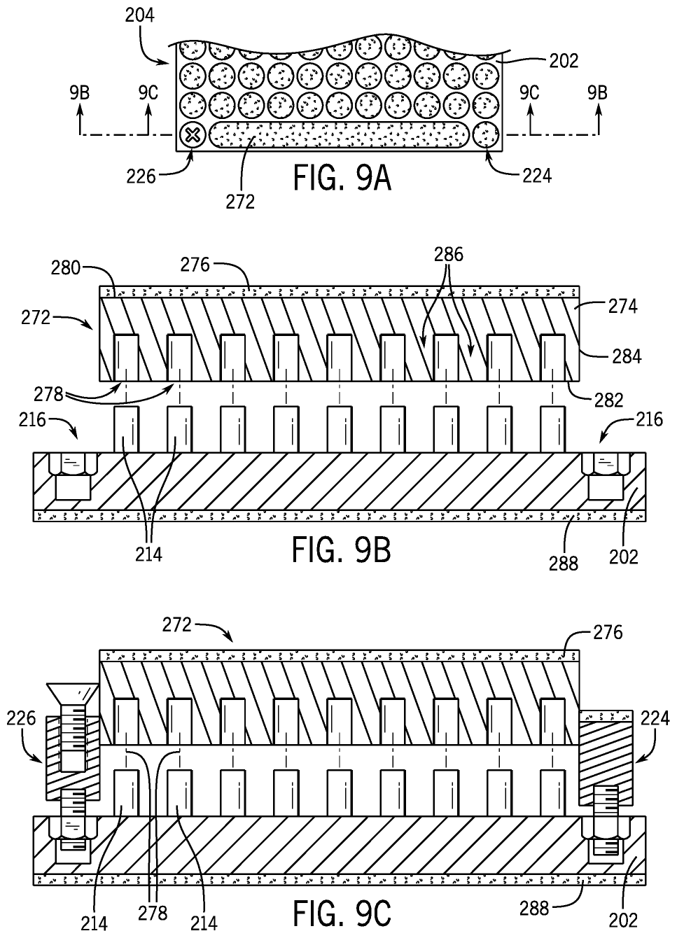

FIGS. 9A-9C shows support columns 204 coupled (and which can be removably coupled) to the base 202, and shows another example of a nesting support column 272, which is a specific non-limiting implementation of the nesting support column 222. The nesting support column 272 includes a body 274, a coupling layer 276, and recesses 278. The body 274 includes an upper surface 280, an opposite lower surface 282, and an adjacent surface (or surfaces) 284 defined between the upper surface 280 and the lower surface 282. As shown, recesses 278 are inwardly directed into the lower surface 282 of the body 274 of the nesting support column 272. Each of the recesses 278 interface with a corresponding peg 214. The width of a particular nesting support column 272 can be greater than the width of two adjacent pegs. In the illustrated embodiment, the nesting support column 272 has nine recesses 278 which each correspond to a peg 214, however, in alternative embodiments the nesting support column 272 may have more or less recesses 278. The nesting support column 272 also includes extensions 286 that are received between adjacent pegs 214. In the illustrated embodiment, the extensions 286 are the same axial height as the pegs 214, however, in alternative embodiments, the axial height of the extensions 286 may be less than (or greater than) the axial height of the pegs 214.

FIG. 9C specifically shows the nesting support column 272 being received on the corresponding pegs 214, a locking support column 226 being received within and threadingly engaging a threaded bore 216 of the base 202, and a fastening support column 224 being received within and threadingly engaging another threaded bore 216.

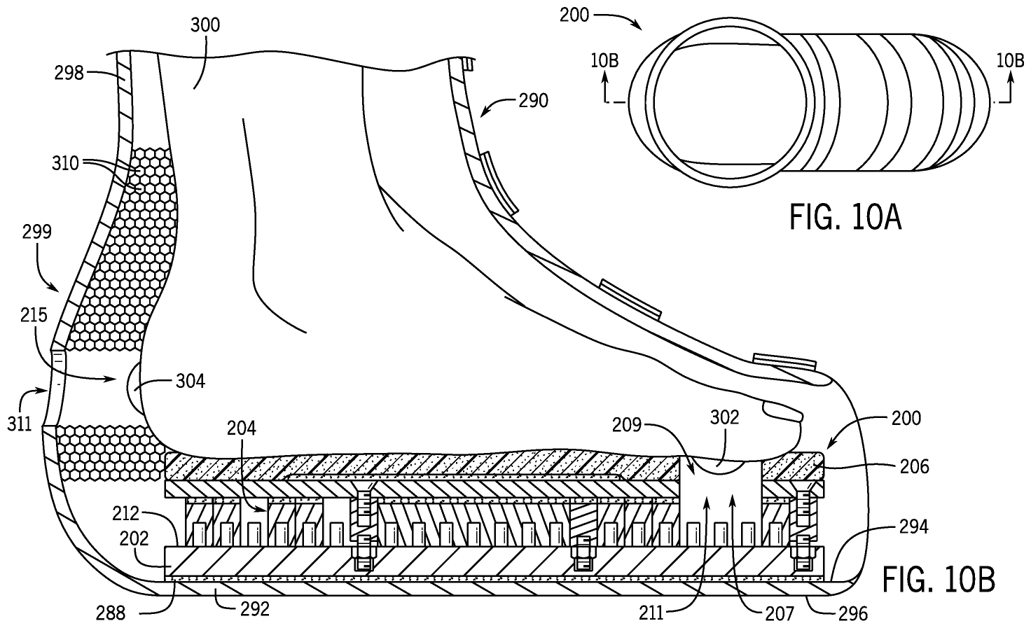

FIGS. 10B and 10A show an example of the orthopedic insert 200 being received within an orthopedic boot 290. The orthopedic boot 290 can include many of the previously discussed components as described regarding the orthopedic boot 100. For example, the orthopedic boot 290 includes a sole 292 having a first surface 294 and an opposite second surface 296. In some embodiments, the orthopedic insert 200 can be installed within the orthopedic boot 290 (or other footwear, such as sandals, boots, shoes, etc.). For example, as shown, a coupling layer 288 can be connected (or coupled) to a lower surface 210 of the base 202, which can be configured to couple, connect, or removably couple the orthopedic insert 200 within the orthopedic boot 290, such as to the sole 292. The coupling layer 288 can embody many different forms, such as the previously discussed coupling layers (e.g., the coupling layers 230, 242, 276). For example, as described above, the coupling layer 288 can include an adhesive (and a backing) that can adhere the base 202 to the first surface 294 of the sole 292 (e.g., where the second surface 296 is configured to contact the ground, such as a walking surface). As another example, the coupling layer 288 can include a loop fastener or a hook fastener, and the first surface 294 can include the other of the loop fastener or the hook fastener to removably couple the base 202 to the sole 292 (or other component within the orthopedic boot 290, such as an intermediate insole layer).

FIG. 10B shows the orthopedic insert 200 being in an installed configuration. For example, the base 202 is coupled to the sole 292, the support columns 204 are removably coupled to the base 202, the insole 206 is coupled to the support column 214, and the cushioning layer 208 is coupled to the insole 206. In some cases, the lower surface of the insole 206 can be coupled, connected, or removably coupled to the support columns 204. For example, the coupling layers of the support columns 204 can have a loop fastener and a hook fastener, and the lower surface of the insole 206 can include the other of the loop fastener and the hook fastener. Similarly, the upper surface of the insole 206 can include a loop fastener and a hook fastener, and the lower surface of the cushioning layer 208 can include the other of the loop fastener and the hook fastener. In some cases, the cushioning layer 208 is adhered to the upper surface of the insole 206 (e.g., via adhesive, etc.).

In some embodiments, the orthopedic insert 200 (and the orthopedic boot 100) are configured to offload pressure (e.g., completely, or decreasing to some extent) from a region that contains a sore, or a region that may be prone to sore formation. Once the desired contact region, and pressure profile is determined, including determining regions that are desired to have minimal loading pressure, the number and different types of support columns 204 can be selected for installation onto the base 202. For example, peripheral regions of the base 202 may be desired to have locking support columns 226, whereas the internal (or central) region of the base 202 may be desired to have nesting support columns 222 and fastening support columns 224. Once the number any type of support columns 204 are chosen, the support columns 204 can be coupled to the base 202. As discussed in more detail below, the regions that are desired to have minimal loading pressure, such as a pressure sore 302 of a foot 300 of a subject do not contain support columns 204.

In some embodiments, and as illustrated, the insole 206 includes an insole aperture 207, and the cushioning layer 208 includes a cushioning layer aperture 209. The insole aperture 207 and the cushioning layer aperture 209 at least partially overlap. For example, in the illustrated embodiment of FIG. 10B, the insole aperture 270 completely overlaps with the cushioning layer aperture 209. In other words, the insole aperture 207 and the cushioning layer aperture 209 are the same shape and size, however in alternative embodiments, the insole aperture 207 and the cushioning layer aperture 209 can be sized and shaped differently. Although the insole 206 and the cushioning layer 208 are shown as each having a single aperture (e.g., the insole aperture 207, and the cushioning layer aperture 209), in alternative embodiments the insole 206 and the cushioning layer 208 can have additional apertures. For example, in some cases, as will be described in more detail below, a foot of a subject may have more than one pressure sore (or pressure sore susceptible regions, or combinations of the two). In this case, the first pressure sore (or susceptible region) can be positioned within the insole aperture 207 and the cushioning layer aperture 209, while the second pressure sore (or susceptible region) can be positioned within the second insole aperture and the second cushioning layer aperture (where at least a portion of the second insole aperture and the second cushioning layer aperture overlap).

As shown in FIG. 10B, the pressure sore 302 of the foot 300 is positioned such that a perimeter of the insole aperture 207, the cushioning layer aperture 209, or both, surrounds the pressure sore 302 to prevent contact between the orthopedic boot 290 and the pressure sore 302 of the subject. In some embodiments, the upper surface 212 of the base 202, the insole aperture 207, the cushioning layer aperture 209, and the adjacent surfaces of the support columns 204 can define a void volume 211. In some embodiments, the surfaces of the support columns 204 (e.g., the adjacent surfaces) and the upper surface 212 of the base 202 can define the void volume 211. The void volume 211 is free of support columns 204 and the pressure sore 302 of the foot 300 is received within the void volume 211. This way, the pressure sore 302 does not contact the orthopedic boot 290, and thus the pressure sore 302 can effectively heal without becoming aggravated from contact. As shown in FIG. 10B, and in some embodiments, the height of the support columns 204 are uniform, which can provide stability to the patient, such as preventing rocking (or tilting) of components within the orthopedic boot 290 (e.g., the cushioning layer 208).

In some embodiments, the orthopedic boot 290 can include a support member 298, which is similar and pertains to the support member 102. The support member 298 does not include an expandable shell (e.g., the expandable shell 106). Rather, the support member 298, which interfaces and connects with the sole 292 and includes a concave region 299. In some cases, the concave region 299 has a radius of curvature that is larger than a heel of a subject, so as to prevent contact between the support member 298 and the heel of the subject. The concave region 299 defines an internal volume, and in some cases, such as in the illustrated embodiment of FIG. 10A, the internal volume of the concave region 299 includes a plurality of cushioned pads 310. The cushioned pads 310 are illustrated as being coupled to adjacent cushioned pads 310 (e.g., by hook and loop fasteners, adhesives, etc.). The cushioned pads 310 can be uniform in shape and size, such that the cushioned pads 310 when coupled together form a tessellated pattern. The tessellated pattern significantly reduces (or eliminates) the gaps between adjacent cushioned pads 310 which is undesirable at least because it decreases stability for the foot 300 of the subject.

In some cases, the cushioned pads 310 can be prisms (e.g., octagonal, rectangular prisms), cubes, pyramidal shapes, etc. The cushioned pads 310 can be formed of materials that offer at least some deformability such as, a memory foam, foams, or other orthotic materials (e.g., Plastazote® polyethylene foam, cork, etc.). In some specific non-limiting embodiments, the cushioned pads 310 can be implemented as rectangular prisms (such as of a foam material) and can be stacked in an array without being fastened to each other. A hole can be created (or exist) through the support member 298, such as through the concave region 299. Then, a foot of a subject can be pressed back against the array of stacked cushioned pads 310 until portions of the cushioned pads 310 just outwardly and though the hole, such that the array of cushioned pads 310 contour the heel of the subject, Then, the cushioned pads 310 can be secured in placed (e.g., such as with an adhesive layer, a layer of hook and loop fasteners, etc.), and the portions of the cushioned pads 310 that just outwardly through the array of stacked cushioned pads 310 can be trimmed accordingly. In alternative configurations, the cushioned pads 310 can be inflatable (e.g., with a fluid, such as air) to provide a degree of cushioning.

FIG. 10B shows a heel of a subject having a pressure sore 304. As shown, the cushioned pads 310 are removed in the region of the pressure sore 304. More specifically, the cushioned pads 310 and the support member 298 define a void volume 215. This way, when the foot 300 is secured within the orthopedic boot 290, the pressure sore 304 is situated within the void volume 215, such that the pressure sore 304 does not contact the cushioned pads 310 and the support member 298. Although FIG. 10A illustrates the support member 298 with the concave region 299, in alternative embodiments, the expandable shell 106 can be substituted with the concave region 299. As such, the cushioned pads 310 can then be received within the expandable shell 106. In alternative configurations, a hole 311 introduced into the support member 298 (or the expandable shell 106) to further define the void volume 215. In this case, the hole 311 can prevent (or mitigate the risk) of the support member 298 (or expandable shell 106) contacting the pressure sore 304.

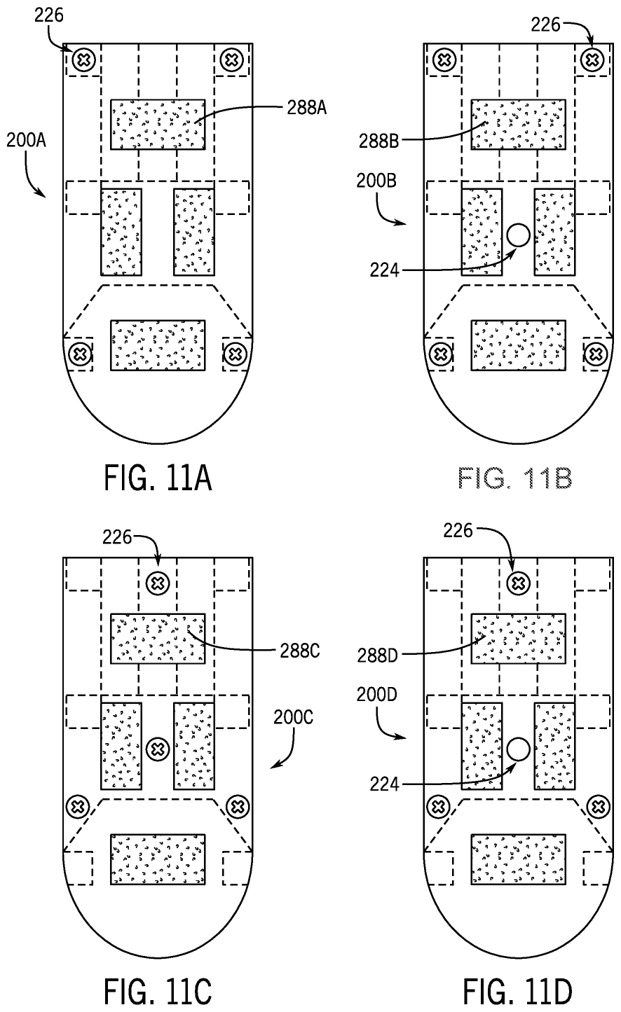

FIGS. 11A-11D show top views of examples of differing orthopedic inserts 200A, 200B, 200C, and 200D. FIGS. 11A-11D have removed cushioning layer for visual clarity. Each of the orthopedic inserts 200A, 200B, 200C, and 200D are specific non-limiting implementations of the orthopedic insert 200 and the previous description of the orthopedic insert 200 pertains to the description of the orthopedic inserts 200A, 200D, 200C, and 200D.

FIG. 11A shows a top view of the orthopedic insert 200A, showing the locations of the locking support columns 226. The locking support columns 226 are located near vertices of the insole 206. For example, if an imaginary rectangle were superimposed over the insole 206, locking support columns 226 would be located at (or placed on) each vertex of the superimposed imaginary rectangle. FIG. 11A also shows a specific non-limiting implementation of a coupling layer 288A, which is shown as patches or regions of a hook fastener, a loop fastener, or a combination of the two configured to be removably coupled to the cushioning layer 208A (not shown).

FIG. 11B shows a top view of the orthopedic insert 200B, which is identical to the orthopedic insert 200A except for a centrally located fastening support column 224 located within a perimeter defined by the locking support columns 226 (e.g., the “imaginary rectangle”). FIG. 11B also shows a specific non-limiting implementation of a coupling layer 288B, which is shown as patches or regions of a hook fastener, a loop fastener, or a combination of the two configured to be removably coupled to the cushioning layer 208B (not shown).

FIG. 11C shows a top view of the orthopedic insert 200C, showing locations of the locking support columns 226. The locking support columns 226 are arranged so that if an imaginary triangle were superimposed over the insole 206, the locking support columns 226 would be located at (or placed on) each vertex of the superimposed imaginary triangle and one locking support column 226 located within the perimeter of the superimposed imaginary triangle. FIG. 11C also shows a specific non-limiting implementation of a coupling layer 288C, which is shown as patches or regions of a hook fastener, a loop fastener, or a combination of the two configured to be removably coupled to the cushioning layer 208C (not shown).

FIG. 11D shows a top view of the orthopedic insert 200D, which is identical to the orthopedic insert 200C except that the centrally located locking support column 226 is replaced with a fastening support column 224. FIG. 11D also shows a specific non-limiting implementation of a coupling layer 288D, which is shown as patches or regions of a hook fastener, a loop fastener, or a combination of the two configured to be removably coupled to the cushioning layer 208D (not shown).

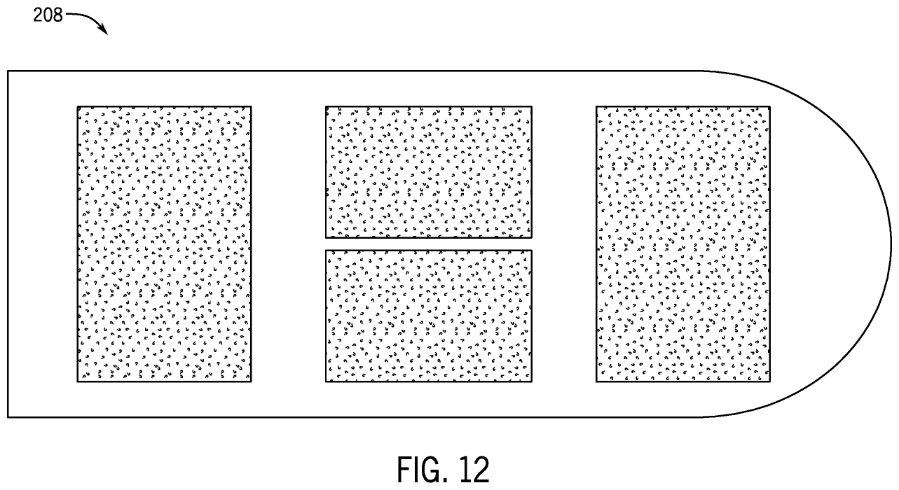

FIG. 12 shows an example of a lower surface of the cushioning layer 208 of the orthopedic insert 200. As shown, the lower surface of the cushioning layer 208 has patches (illustrated as rectangles) of a hook fastener, a loop fastener, or a combination of the two, which is configured to be removably coupled to the insole 206.

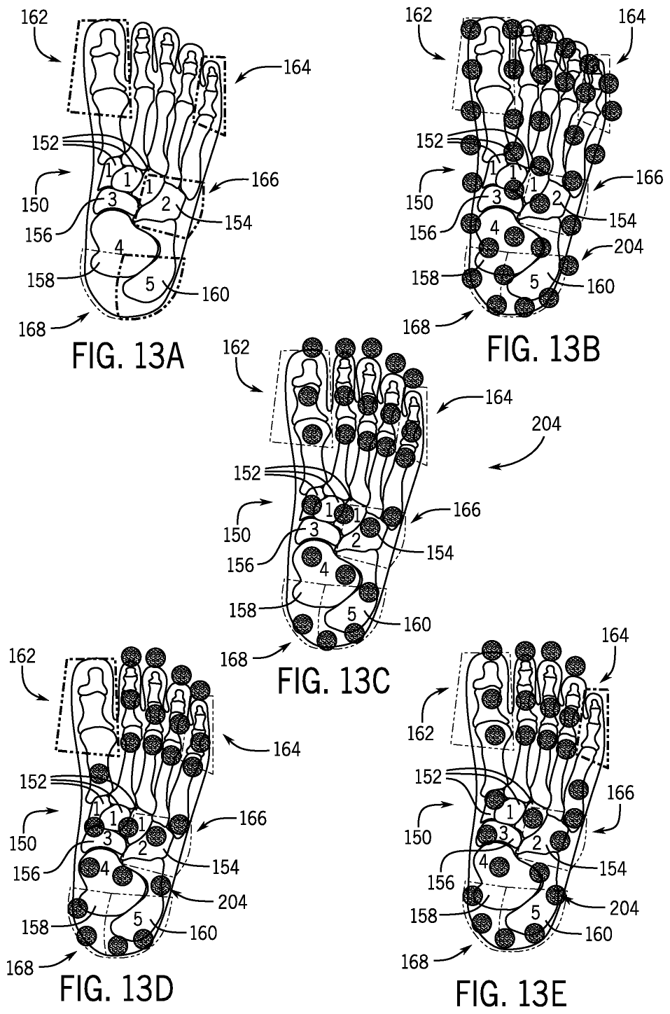

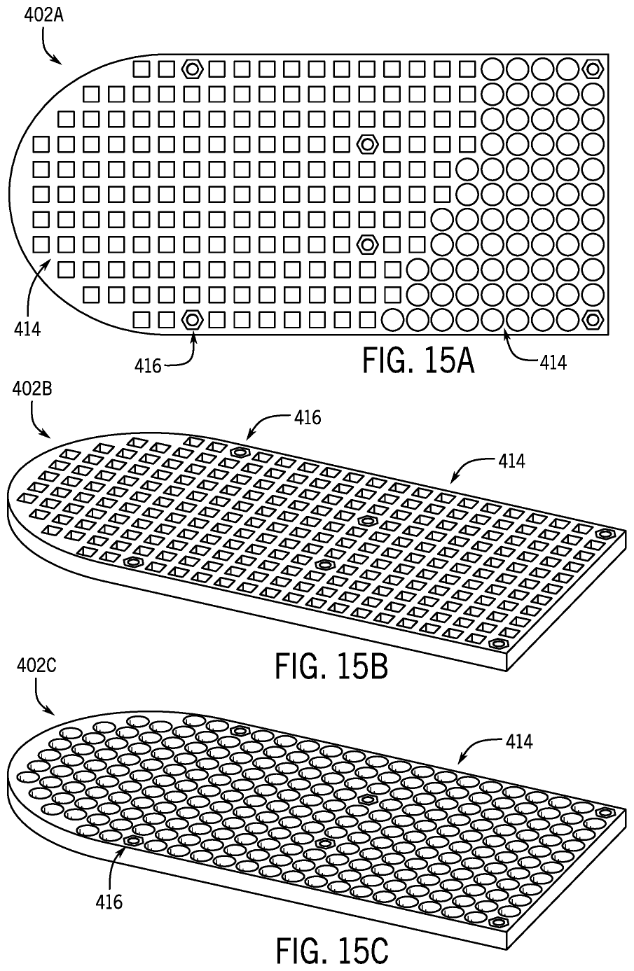

FIGS. 13A-13I show top view diagrams of anatomical structures (e.g., bones) of a foot of a subject having overlaid support columns (e.g., support columns 110, 204, 404, 504), showing the relative position or configurations of the support columns to the anatomical structures. Each of FIGS. 16A-16I shows a diagram 150 of anatomical structures of a foot. The diagrams 150 show the cuneiforms 152, the cuboid 154, the navicular 156, the talus 158, and the calcaneus 160. The diagram 150 also shows regions 162, 164, 166, 168 that, in some cases, may be prone to pressure sore (or wound) formation.

FIG. 13A shows a diagram of a foot of a subject with labeled anatomical structures of the foot, and labeled regions 162, 164, 166, 168 that may be prone to pressure sore formation. In some cases, the practitioner can either place or refrain from placing (or installing) support columns 204 on or near the anatomical structures 152, 154, 156, 158, 160, or on or near the regions 162, 164, 166, 168. In some cases, pressure sores (or other wounds) are more likely to form over bony prominences, such as the anatomical structures 152, 154, 156, 158, 160. Thus, in these cases, the support columns 204 are not placed over anatomical structures or within the regions 162, 164, 166, 168.

FIG. 13B shows a diagram of a foot of a subject of FIG. 13A with support columns in a confirmation, overlaid on the diagram. As shown in FIG. 13B, the anatomical structures 152, 154, 156, 158, 160, and the regions 162, 164, 166, 168 all include support columns 204. In the configuration of FIG. 13B, the support columns 204 are arranged such that no plantar decompression is required.

FIG. 13C shows another diagram of a foot of a subject of FIG. 13A with support columns in another confirmation, overlaid on the diagram. As shown in FIG. 13C, support columns 204 are strategically placed over some of the anatomical structures.

FIG. 13D shows another diagram of a foot of a subject of FIG. 13A with support columns in another confirmation, overlaid on the diagram. As shown in FIG. 13D, support columns 204 are strategically placed over some of the anatomical structures, and no support columns 204 are located within the region 162, which includes at least a portion of the hallux (e.g., the big toe of the subject).

FIG. 13E shows another diagram of a foot of a subject of FIG. 13A with support columns in another confirmation, overlaid on the diagram. As shown in FIG. 13E, support columns 204 are strategically placed over some of the anatomical structures, and no support columns 204 are located within the region 164, which includes at least a portion of the outermost toe (or fifth toe), (e.g., the little toe of the subject).

FIG. 13F shows another diagram of a foot of a subject of FIG. 13A with support columns in another confirmation, overlaid on the diagram. As shown in FIG. 13F, support columns 204 are strategically placed over some of the anatomical structures, and no support columns 204 are located within the region 168, which includes at least a portion of the talus 158, and the calcaneus 160 of the subject (e.g., at least a portion of the heel of the subject).

FIG. 13G shows another diagram of a foot of a subject of FIG. 13A with support columns in another confirmation, overlaid on the diagram. As shown in FIG. 13G, support columns 204 are strategically placed over some of the anatomical structures, and no support columns 204 are located within the region 166, which includes at least a portion of the cuboid 154 of the subject.

FIG. 13H shows another diagram of a foot of a subject of FIG. 13A with support columns in another confirmation, overlaid on the diagram. As shown in FIG. 13H, support columns 204 are strategically placed over some of the anatomical structures, and no support columns 204 are located within any of the regions 162, 164, 166, 168.

FIG. 13I shows another diagram of a foot of a subject of FIG. 13A with support columns in another confirmation, overlaid on the diagram. As shown in FIG. 13I, support columns 204 are strategically placed, such that no support columns 204 are located within the regions 162, 164, 166, 168, and no support columns 204 are located over any of the anatomical structures 152, 154, 156, 158, 160.

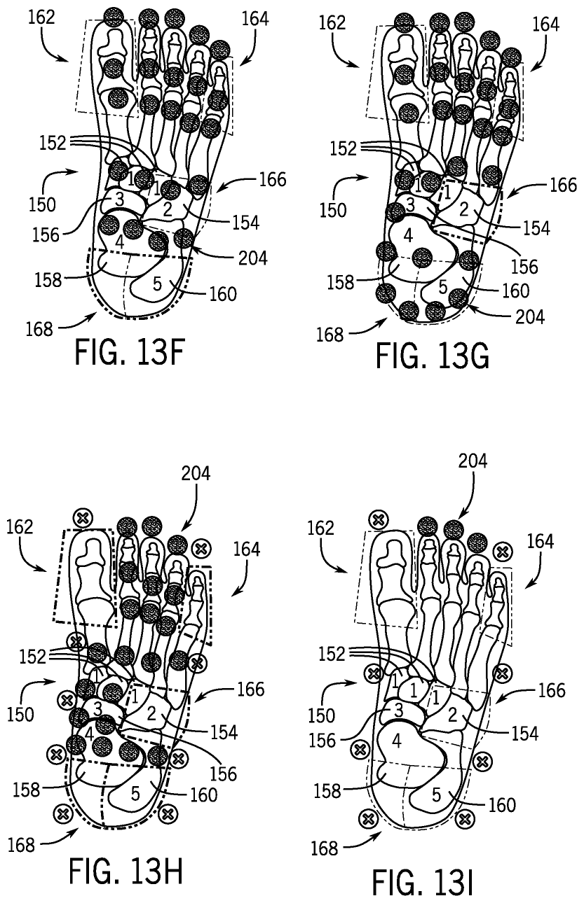

FIGS. 14A and 14B shows an example of another orthopedic insert 400 according to some embodiments of the disclosure. The orthopedic insert 400 is similar to the orthopedic insert 200, and thus the discussion of the orthopedic insert 200 pertains to the orthopedic insert 400. The orthopedic insert 400 also includes a base 402, support columns 404, an insole 406, and a cushioning layer 408. The base 402 includes a lower surface 410, an opposite upper surface 412, and recesses 414 downwardly extending into the upper surface 412 of the base 402. As will be described in more detail below, the support columns 404 are removably coupled to the corresponding recesses 414.

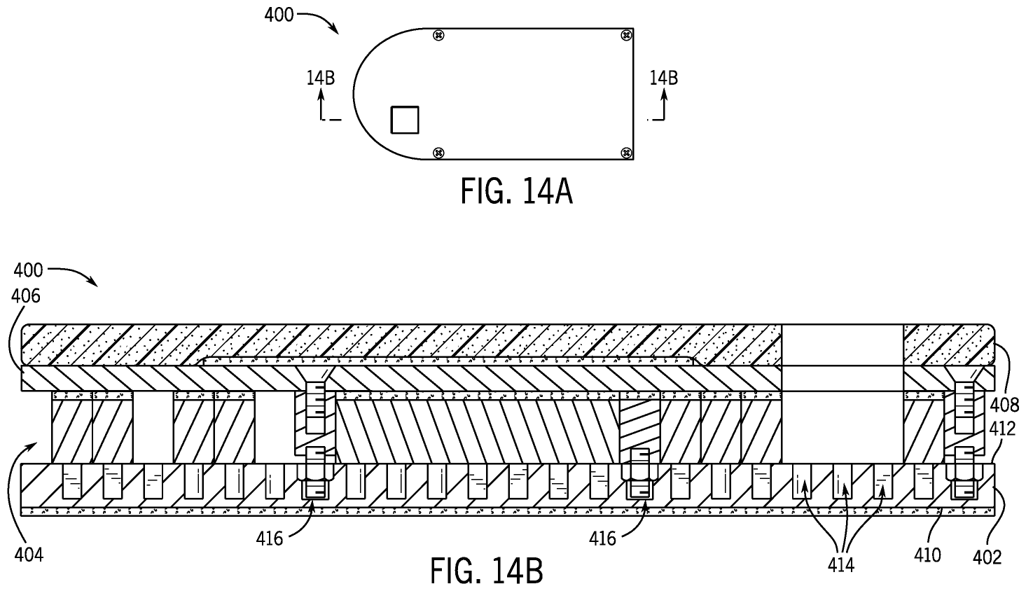

FIGS. 15A-15C show an example of bases 402A, 402B, 402C, as alternatives to the base 402 of the orthopedic insert 400. The recesses 414 correspond to the shape of the protrusions of (some of) the support columns 404. As shown, the recesses 414 can be rectangular in shape, and can be cylindrical in shape. In some cases, the recesses 414 can embody other shapes, such as prisms (e.g., triangular prisms, rectangular prisms, octagonal prisms, combinations thereof, etc.) In some embodiments, the recesses 414 can all be the same shape and size. FIGS. 15A-15C also show threaded bores 416 directed into the upper surface of the respective base.

FIGS. 16A-16C show an example of various support columns 404 being coupled to the base 402. The support columns 404 include interfacing support columns 422. The interfacing support columns 422 can include a body 428, a coupling layer 430, and a protrusion 432. The body 428 includes an upper surface 434, a lower surface 436, and an adjacent surface (or surfaces) 438 defined between the upper surface 434 and the lower surface 436. The protrusion 432 extends from the lower surface 436 of the body 428, and can be coupled, connected, or integrally formed with the body 428. In some embodiments, the protrusion 432 can be formed of various rigid materials (e.g., plastics, metals, etc.). As shown in FIG. 16A, the protrusion 432A can be rectangularly shaped, and in FIG. 16B, the protrusion 432B can be cylindrically shaped. To install the interfacing support columns 422, the protrusion 432 is inserted into a correspondingly shaped recess 414. In some embodiments, and as described previously the support columns 404 include fastening support columns 424 and locking support columns 426.

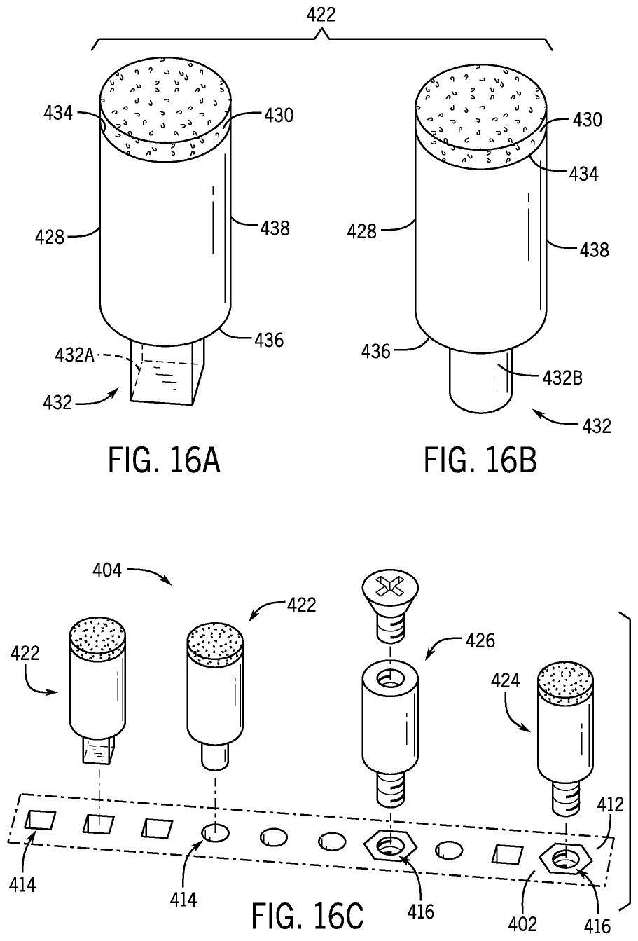

FIGS. 17A and 17B show another example of an interfacing support column 472, which is a specific non-limiting implementation of the interfacing support column 422. The interfacing support column 472, includes a body 474, a coupling layer 476, and protrusions 478. The body 474 includes an upper surface 480, an opposite lower surface 482, and an adjacent surface (or surfaces) 484 defined between the upper surface 480 and the lower surface 482. As shown, the protrusions 478 are similar to the protrusion 432 in that they extend from the lower surface 482 of the interfacing support column 472. The protrusions 478 can be coupled, connected, or integrally formed with the body 474. As shown, the protrusions 478 correspond to the shape (and size) of the recesses 414. As discussed previously, the protrusions 478 and the recesses 414 can be implemented as rectangular prisms, or can be implemented as cylinders. In some embodiments, such as illustrated in FIG. 16A, on the same interfacing support column 472, some of the protrusions 478 can be rectangular prisms, while some of the protrusions 478 can be cylinders. Thus, correspondingly some of the recesses 414 can be cylinders and some of the recesses 414 can be rectangular prisms. FIG. 16B also shows a locking support column 426 and a fastening support column 424, each interfacing with a given threaded bore 416.

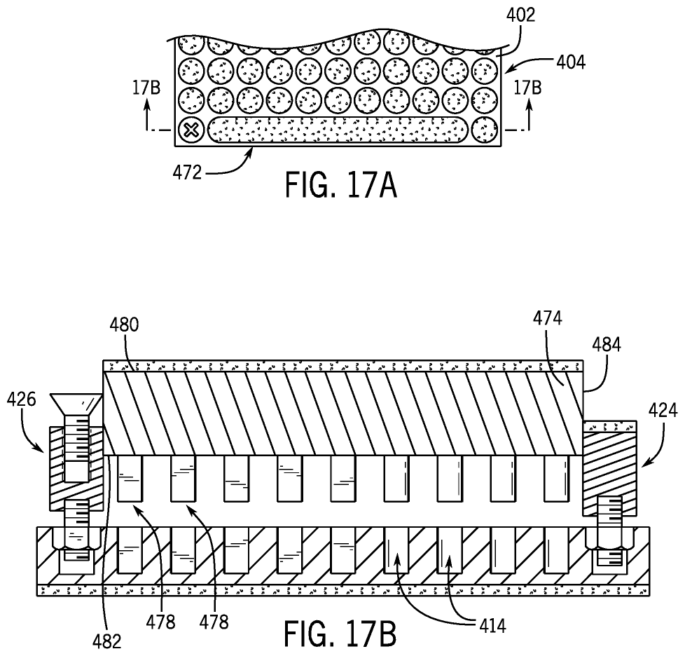

FIGS. 18A and 18B show the orthopedic insert 400 in an installed configuration. For example, the support columns 404 are coupled to and interface with the base 402. The insole 406 is connected to the support columns 404 via the coupling layers (or fasteners) of the support columns 404. As shown, the insole 406 includes an insole aperture 407, and the cushioning layer 408 includes a cushioning layer aperture 409. The insole aperture 407 and the cushioning layer aperture 409 at least partially overlap. For example, in the illustrated embodiment of FIG. 17A, the insole aperture 407 completely overlaps with the cushioning layer aperture 409. In other words, the insole aperture 407 and the cushioning layer aperture 409 are the same shape and size, however in alternative embodiments, the insole aperture 407 and the cushioning layer aperture 409 can be sized and shaped differently. Although the insole 406 and the cushioning layer 408 are shown as each having a single aperture (e.g., the insole aperture 407, and the cushioning layer aperture 409), in alternative embodiments the insole 406 and the cushioning layer 408 can have additional apertures.

In some embodiments, the upper surface 412 of the base 402, the insole aperture 407, the cushioning layer aperture 409, and the adjacent surfaces of the support columns 404 can define a void volume 411. In some embodiments, the surfaces of the support columns 404 (e.g., the adjacent surfaces) and the upper surface 412 of the base 402 can define the void volume 411. The void volume 411 is free of support columns 404 and a pressure sore of a foot is received within the void volume 411. This way, the pressure sore does not contact any portion of the orthopedic insert 400, and thus the pressure sore an effectively heal without becoming aggravated from contact. As shown in FIG. 18B, and in some embodiments, the height of the support columns 404 are uniform, which can provide stability to the patient, such as preventing rocking (or tilting) of components within the orthopedic insert 400.

Similarly to the description of FIG. 10B, FIG. 18B shows a foot with pressure sores received within an orthopedic boot having the orthopedic insert 400. As previously described, the orthopedic insert 400 could be inserted into a number of different footwear options, such as, for example, shoes, boots, sandals, etc. As shown, a pressure sore of a subject located within the void volume 411. The orthopedic boot also includes a plurality of cushioned pads (e.g., cushioned pads 418). The cushioned pads and a convex region (or aperture) of a support member of the orthopedic boots define another void volume, which can receive a pressure sore (or other appropriate region) 415 of a heel of a subject.

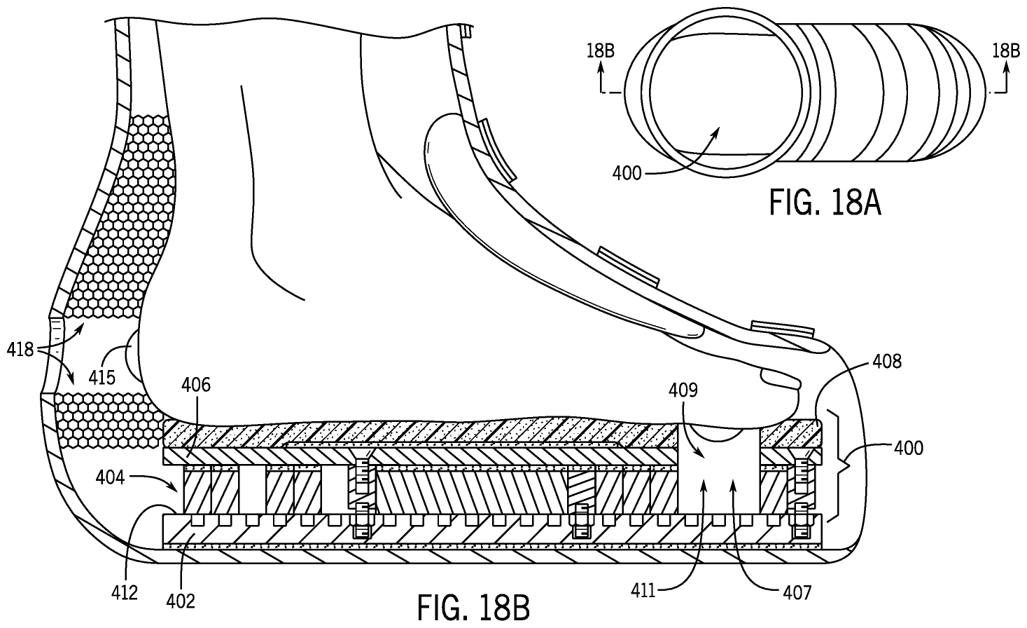

FIGS. 19A-19C show another example of an orthopedic insert 500 according to some embodiments of the disclosure. The orthopedic insert 500 is similar to the previously described orthopedic inserts 200, 400. For example, the orthopedic insert 500 includes a base 502, support columns 504, an insole 506, and a cushioning layer 508. The base 502 includes a lower surface 510, an opposite upper surface 512. As will be described in more detail below, the support columns 504 are removably coupled to the base 502.

The support columns 504 include a body 514 having an upper surface 516, a lower surface 518, and adjacent surfaces 520 defined between the upper surface 516 and the lower surface 518. The lower surface 518 of the support column 504 includes a coupling layer 522, which is similar to the previously discussed coupling layers (e.g., coupling layer 230, coupling layer 242, coupling layer 276, coupling layer 288, coupling layer 430, etc.). For example, in some cases, the coupling layer 522 of the body 514 of the support column 504 can be implemented as a hook fastener or a loop fastener. In this case, the upper surface 512 of the base 502 can have the other of the hook fastener or loop fastener. This way, the support columns 504 can be easily placed and removed from the base 502 so as to easily create a void that allows a portion of a subject's foot (e.g., a pressure sore) to significantly reduce contact (or entirely reduce contact) with a portion of the orthopedic insert 500, as will be discussed in more detail below.

Although the removably coupled nature of the support columns 504 allows for easier removal of the support columns 504 from the base 502 (e.g., to form a void having a void volume), in other configurations, the coupling layer 522 can be implemented as an adhesive such as with a film backing. This way, after determining how many, the type (e.g., size, thickness, shape, etc.), and number of support columns 504 are desired. The lower surface 518 of the support columns 504 can be adhered to the upper surface 512 of the base 502 (such as after removing the film backing). In some cases, such as in this configuration, the support columns 504 can be packaged in an array (e.g., a square of 10 by 10). This way, a film backing can be removed, which covers the array of support columns 504 and exposes the adhesive of each of the coupling layers 522 of the support columns 504, and the array of support columns 504 can be adhered to the upper surface 512 of the base 502 all at once. In some cases, a film can be wrapped around the adjacent surfaces 520 of the array of support columns 504 which can aid in installation of the array of support columns 504, such that after the array of support columns 504 is adhered to the upper surface 512 of the base 502, the film can be removed.

In some embodiments, the coupling layer 522 support column 504 can magnetically couple (and removably couple) the support column 504 to the upper surface 512 of the base 502. For example, in some cases, the coupling layer 522 can include a magnet, and the upper surface 512 of the base 502 can include another magnet (e.g., embedded within the base 502, or joined to the surface). This way, when the magnet of the coupling layer 522 of the support column 504 is brought into contact with the another magnet of the upper surface 512 of the base 502, the support column 504 is attracted and coupled to the base 502. In this configuration, for example, any given support column 504 can be easily removed (or installed) on the base 502. For example, the base 502 may already have the support columns 504 coupled thereto, and a number of support columns 504 can be easily removed to create the void (and corresponding void volume).

FIG. 19A shows an example of the support columns 504 coupled to the base 502. A number of support columns 504 have been removed from the base 502 to create a void volume 524. For example, as shown, the adjacent surfaces 520 of the support columns 504, and the upper surface 512 of the base 502 can define the void volume 524. In some cases, such as when the insole 506 includes an insole aperture 507 directed through the insole 506, and a cushioning layer 508 includes a cushioning layer aperture 509 directed through the cushioning layer 508, the insole aperture 507, the cushioning layer aperture 509, the adjacent surfaces 520 of the support columns 504, and the upper surface 512 of the base can define the void volume 524. As shown in FIG. 19B, the orthopedic insert 500 also includes a void volume 526. Unlike the void volume 524 which is rectangular shaped and is defined by the unmodified adjacent surfaces 520 of the support columns 504, the void volume 526 is defined by modified adjacent surfaces 520 of the support columns 504. For example, in some cases, the void volume 526 is created by cutting (such as stamping) a desired size and shape of a void volume through the cushioning layer 508, the insole 506, the support columns 504, independently or in combination, to create the void volume 526.

As shown in FIGS. 19A-19C, the support columns 504 all have the same axial height. In alternative embodiments, the support columns 504 can have differing axial heights, such as a topographical landscape so as to create the desired loading (or pressure) profile on the foot of the subject. In some cases, the support columns 504 can be shaped differently, such as having other tessellated patterns (e.g., octagonal prisms, triangular prisms, cubes, etc.). As shown, and in these tessellated alternative configurations, the support columns 504 are configured to contact each other (e.g., adjacent surfaces 520 of adjacent support columns 504 contact each other). In some cases, the support columns 504 can have different axial heights, different cross-sectional areas, and can be formed of different materials (such as when all of the support columns 504 are identical) to align with the desired pressure profile or loading profile directed on the foot of the subject. In some cases, the support columns 504 can be formed out of various orthotic materials that can have some deformability (e.g., foams, Plastazote® polyethylene foam, cork, etc.).

FIG. 19C shows a boundary 528, which can be the shape of a typical insert for various types of footwear. In some cases, the boundary 528 can be formed by cutting (e.g., stamping) out the desired shape from the assembled orthopedic insert 500.

As discussed above, all of the bases of the orthopedic inserts 200, 400, 500 can be substituted for a sole of a boot, shoe, etc., where the substituted sole includes the components of the respective bases of the orthopedic inserts 200, 400, 500. This way, embodiments of the previously discussed orthopedic inserts can be implemented on various types of footwear, such as sandals, boots (e.g., orthopedic boots), shoes, etc. Additionally, although the orthopedic inserts 200, 400, 500 have different features, any embodiments within the orthopedic inserts 200, 400, 500 can be interchanged with other embodiments. For example, another orthopedic insert (or sole, such as part of a shoe) can have both pegs 214, recesses 414, a coupling layer (e.g., as in the embodiment of the orthopedic insert 500), or any suitable combination.

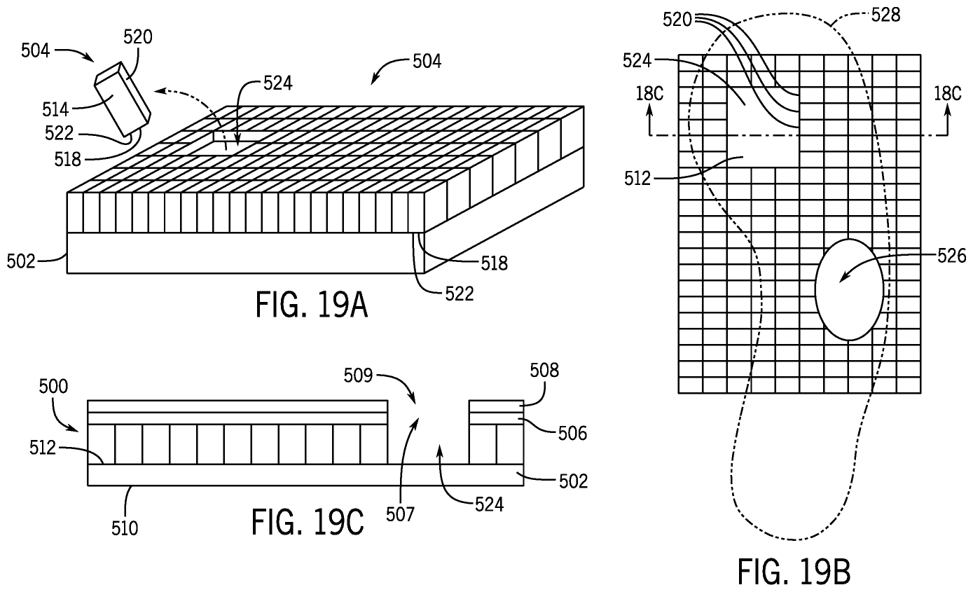

FIG. 20B shows a cross-section taken along the sagittal plane of an example of another orthopedic insert 600 installed within footwear (e.g., an orthopedic boot), according to some embodiments of the disclosure. Similarly to the other previously described orthopedic inserts, the orthopedic insert 600 includes a base 602, support columns 604, an insole 606, and a cushioning layer 608. In some embodiments, the support columns 604 are removably coupled to the base 602. Each of the support columns 604 can include a body 610 having an upper surface 612, a lower surface 614, an adjacent surface (or surfaces) 615 defined between the upper surface 612 and the lower surface 614. A horizontal axis 616 is overlaid on the sagittal plane cross-section to show the features of the support columns 604.

As shown, the upper surfaces 614 of the support columns 604 are angled relative to the horizontal axis 616, when viewed in the sagittal plane. In other words, a surface 614 of a support column 604 and the horizontal axis define an angle. In some cases, the angle can be 1 degree to 20 degrees, 1 degree, 2 degrees, 4 degrees, 6 degrees, 8 degrees, etc. In some embodiments, each support column 604 (or wedges as described below) can have a height (e.g., of eight centimeters, 4 centimeters, 2 centimeters, etc.). More specifically, in some cases, the upper surfaces 614 of the support columns 604 are angled downwardly relative to the horizontal axis 616 at the angle. In this case, the insole 606 is coupled (or removably coupled) to the support columns 604, such that when the foot is secured within the foot wear and supported by the orthopedic insert 600, the foot (and the insole 606) is angled along the angle defined by the support columns 604. This angled configuration (e.g., downward) can be advantageous, because it can equalize the loading pressure of the foot, while having the foot positioned in plantar flexion stimulating the ruptured Achilles tendon to heal. In some embodiments, the support columns 604 can include a single or plurality of recesses (e.g., recesses 278), a plurality of or protrusions (e.g., protrusions 478), a single recess (e.g., recess 232), or a single protrusion (e.g., protrusion 432).

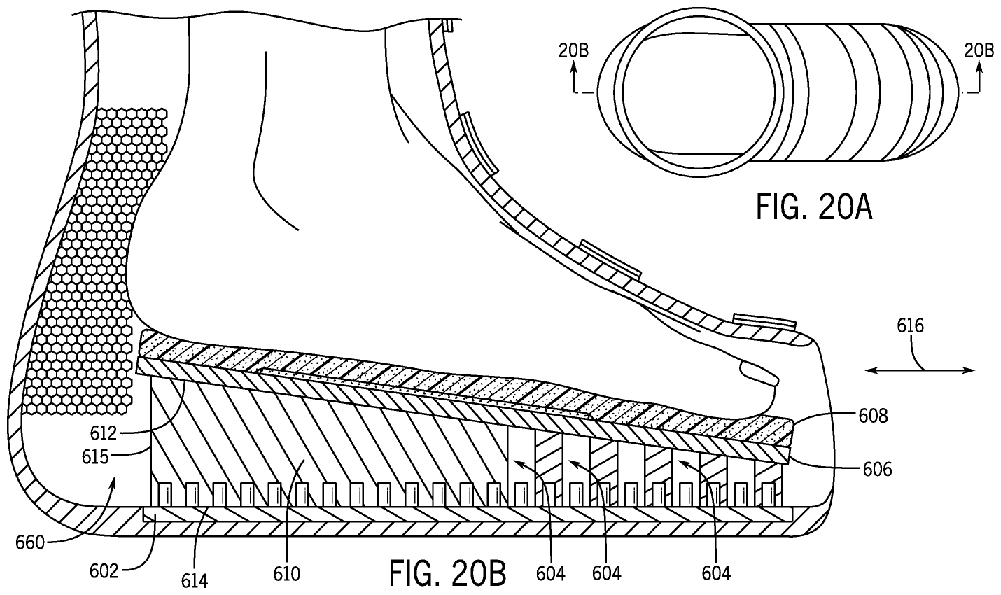

FIGS. 21A and 21B shows a cross-section taken along the sagittal plane of an example of another orthopedic insert 600A installed within footwear (e.g., an orthopedic boot), according to some embodiments of the disclosure. Similarly to the other previously described orthopedic inserts, the orthopedic insert 600A includes a base 602A, support columns 604A, an insole 606A, and a cushioning layer 608A. In this embodiment, the support columns 604A are removably coupled to the base 602A. Each of the support columns 604A can include a body 610A having an upper surface 612A, a lower surface 614A, an adjacent surface (or surfaces) 615A defined between the upper surface 612A and the lower surface 614A. A horizontal axis 616A is overlaid on the sagittal plane cross-section to show the features of the support columns 604A. The orthopedic insert 600A also includes wedges 617A that are configured to be removably coupled (e.g., via Velcro® fastener) or coupled, or connected to the support columns 604A, or in some cases and as illustrated, to other wedges 617A (e.g., each other). The wedges 617A have a upper surface 618A, an opposite lower surface 620A, and adjacent surfaces 621A defined between the upper surface 618A, and the lower surface 620A. The lower surface 620A, and the upper surface 618A of the wedges 617A can determine how the wedges 617A interface with the support columns 604A, other wedges 617A, and the insole 606A. For example, and as illustrated in FIG. 21B, an upper surface of the support column 604 is angled at an angle relative to a horizontal axis 616A overlaid on the sagittal plane (e.g., the cross-sectional view). Additionally, the upper surface 618A and the lower surface 620A of the wedge 617A are angled at the same angle or different angles (or in some cases where one is flat, such as substantially parallel) relative to the horizontal axis 616A of the sagittal plane. This way, an angle of the insole 606A relative to the horizontal axis 616A of the sagittal plane can be adjusted (e.g., to decrease loading on injuries away from the front of the foot, such as Achilles tendon injuries, by moving the center of mass of the foot).

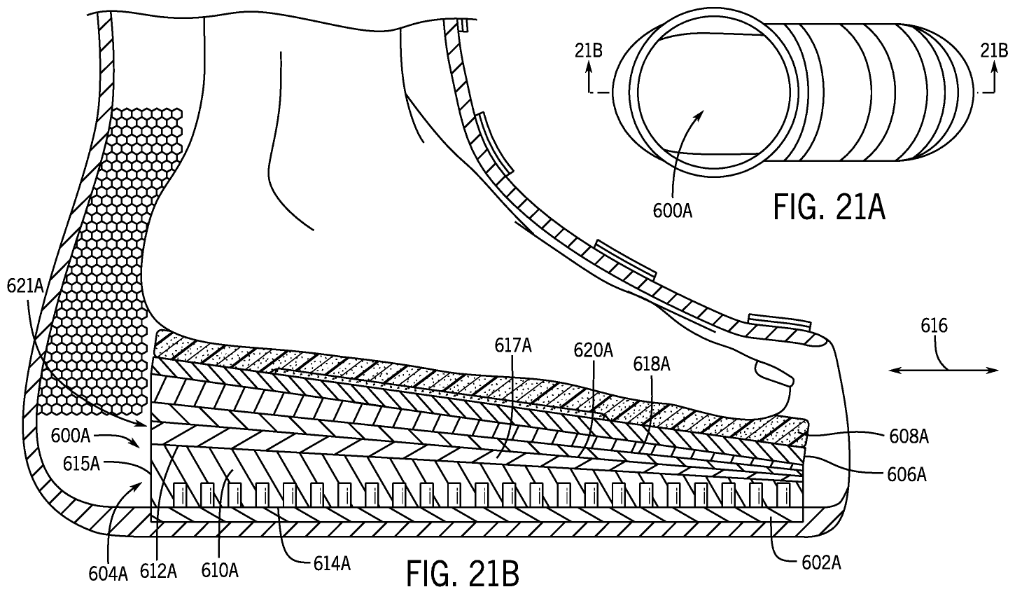

FIG. 22 shows an example of the support column 604A and the wedge 617A. The upper surface 618A of the wedge 617A is downwardly angled relative to a horizontal axis 616A. The support column 604A has an upper surface that is substantially parallel to the horizontal axis 616A. The support column 604A includes a coupling layer 624A that is configured to removably coupled (couple, or connect) the wedge 617A. In some embodiments, the wedges 617A can be formed out of the same material as the previously discussed support columns.

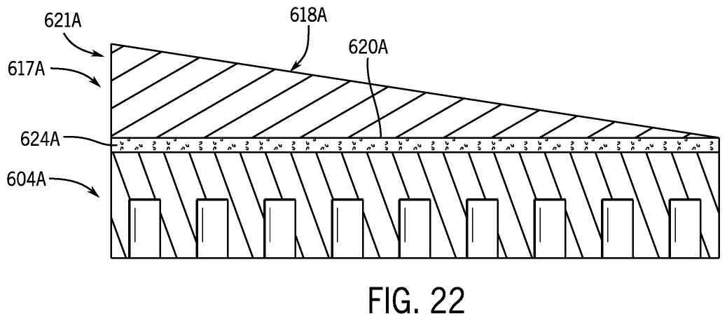

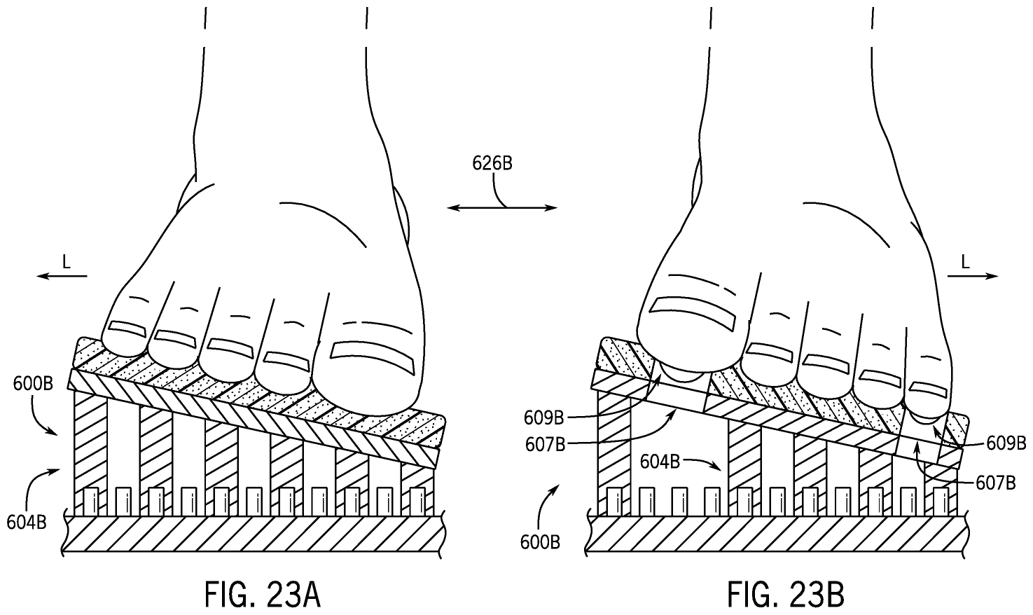

FIGS. 23A, 23B, 24A, and 24B show alternative examples of orthopedic inserts 600B, 600C having respective angled support columns 604B, and angled support columns 604C. Specifically, FIG. 23A shows a coronal plane view (e.g., a front view) of the orthopedic insert 600B having angled support columns 604B. Specifically in FIG. 23A, an upper surface of the support column 604B is angled at an upward angle in a lateral direction L of a transverse axis 626B overlaid on the coronal plane, where the horizontal axis 616A is perpendicular to the transverse axis 626B. FIG. 23B specifically shows insole apertures 607B, and cushioning layer apertures 609B. In FIG. 23B, an upper surface of the support column 604B is angled at a downward angle in a lateral direction L of a transverse axis 626B overlaid on the coronal plane, where the horizontal axis 616A is perpendicular to the transverse axis 626B. FIG. 23B specifically shows insole apertures 607B, and cushioning layer apertures 609B.

FIG. 24A shows a front view of a coronal plane view (e.g., a front view) of the orthopedic insert 600C having angled support columns 604C. Specifically in FIG. 24A, an upper surface of the support column 604C (the insole 606C, and wedges 617C) is angled at a downward angle in a lateral direction L relative to a transverse axis 626C overlaid on the coronal plane, where the horizontal axis 616A is perpendicular to the transverse axis 626C. FIG. 24B specifically shows wedges 617C coupled to the support column 604C that are configured to adjust the angle of the insole 606C relative to the transverse axis 626C. FIG. 24B also shows an upper surface of the support column 604C (the insole 606C, and wedges 617C) being angled at an opposing magnitude of the angle shown in FIG. 24A (e.g., a mirror image of FIG. 24A, where the angle of FIG. 24A is positive, and the angle of FIG. 24B is negative). As such, in FIG. 24B, an upper surface of the support column 604C (the insole 606C, and wedges 617C) can be angled upwardly at an upward angle in the lateral direction of FIG. 24A relative to the transverse axis 626C.

As shown, in FIGS. 23A, 23B, 24A, and 24B, the support columns are implemented to be removably coupled via pegs, however in alternative embodiments other removably coupled configurations such as previously discussed configurations could be used. The angle of the wedges, and support columns, that adjust the angle of the insole relative to the transverse axis, can be used for supporting configurations that require eversion or inversion (e.g., such as foot treatments that call for eversion or inversion). As shown, various wedges 617C with differing angles (E.g., relative to the transverse axis 626C, and the specified lateral directions) can be used to adjust the angle of the insole 606C.

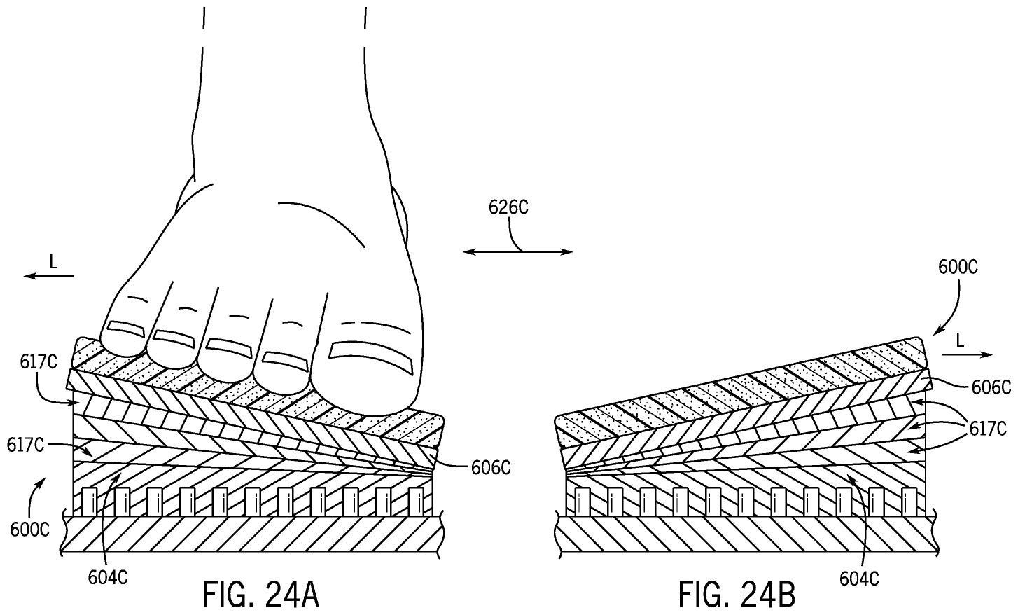

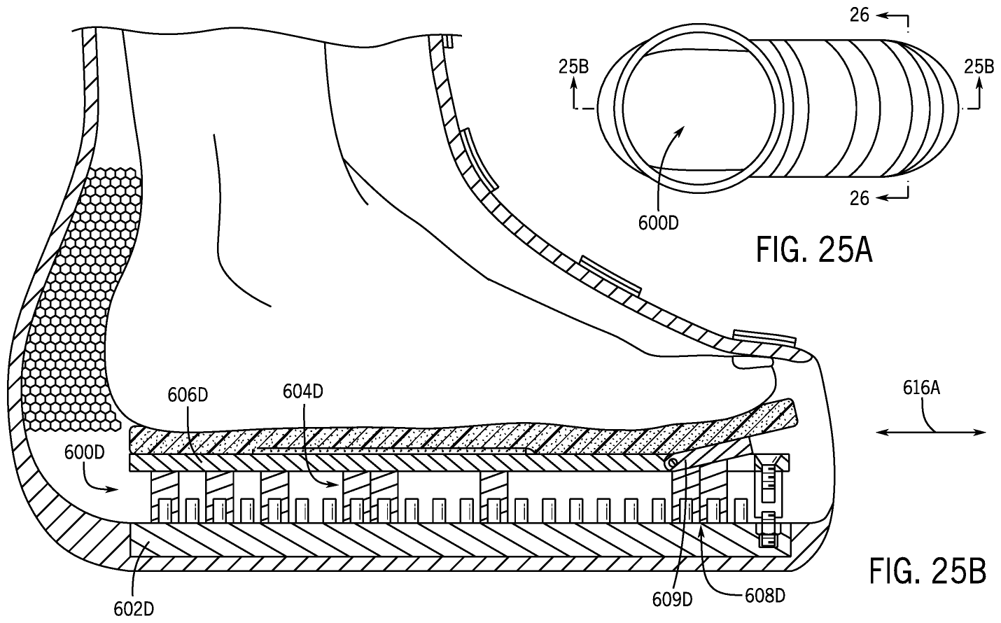

FIGS. 25A, 25B, and 26 show an example of another orthopedic insert 600D, according to some embodiments of the disclosure. The orthopedic insert 600D has support columns 604D, some of which have an upper surface that is angled relative to the horizontal axis 616A. In this configuration, a group of support columns 608D have an upper surface angled relative to the horizontal axis 616A, and are configured to angle a portion of the insole 606D relative to the horizontal axis 616A. For example, a portion 609D of the insole 606D is pivotally (or hingedly) connected (e.g., via pins) to the remaining portion of the insole 606D. This way, the portion 609D of the insole 606D can be set at a desired angle relative to the horizontal axis 622A, for example, by being supported by the group of support columns 608D having the upper surface angled relative to the horizontal axis 616A. The portion 609D of the insole 606D that is pivotally connected can be configured to support a talus (e.g., a big toe) of a subject. In other configurations, the orthopedic insert 600D can include a torsional spring that biases the portion 609D of the insole 606D that is pivotally connected. This way, the group of support columns 608D having the upper surface angled relative to the horizontal axis 622A can be removed in place of the torsional spring (or torsional springs on respective sides of the pins). The torsional spring (or springs) can bias the portion 609D of the insole 606D at an angle relative to the horizontal axis 622A. In this case, if a downward force were directed on the portion 609D of the insole 606D, the portion 609D of the insole 606D would rotate in a clockwise direction (e.g., relative to the view in FIG. 24) towards a base 602D, but would return to its angled positon (e.g., counterclockwise relative to the base 602D) after the downward force was removed.

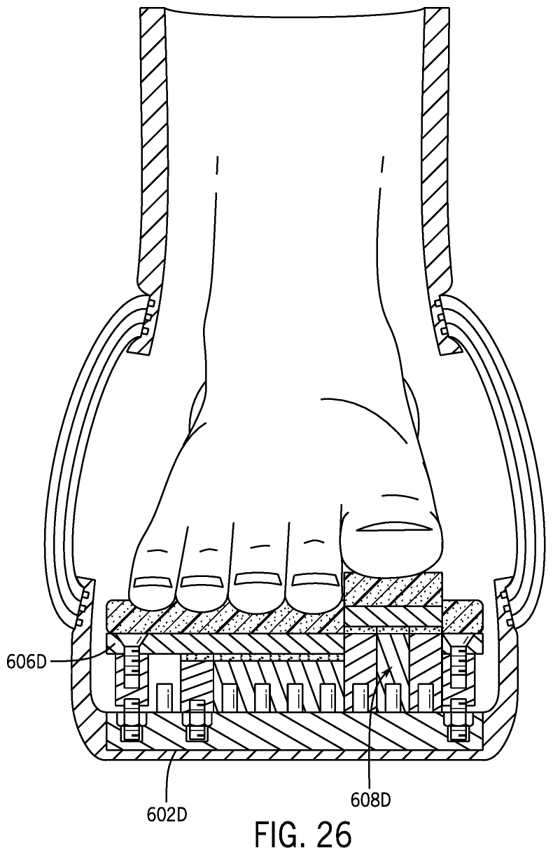

FIGS. 27A and 27B show an example of another orthopedic insert 700 installed within footwear (e.g., the orthopedic boot 290), according to some embodiments of the disclosure. Similarly to the other previously described orthopedic inserts, the orthopedic insert 700 includes a base 702, support columns 704, an insole 706, an insole aperture 707 directed through the insole 706, and a cushioning layer 708. In some embodiments, the support columns 704 are removably coupled to the base 702. As illustrated in FIGS. 27A and 27B, the support columns 704 can be have a first axial height 710, and alternatively, can have a second axial height 712. The second axial height 712 is greater than the first axial height 710, such that the support columns 704 having the second axial height 712 extend though the insole aperture 707. In some cases, such as when the cushioning layer 708 is disposed on the insole 706, the support columns 704 having the second axial height 712 extend through the insole aperture 207 to contact and upwardly deflect a region of the cushioning layer 708. In some cases, such as when a subject has a condition that requires loading or pressure to a region of the foot 714 (such as metatarsalgia, capsulitis, neuromas, sesamoiditits, etc.), the support columns 704 having the second axial height 712 contact and load (or apply pressure) to the region of the foot 714.

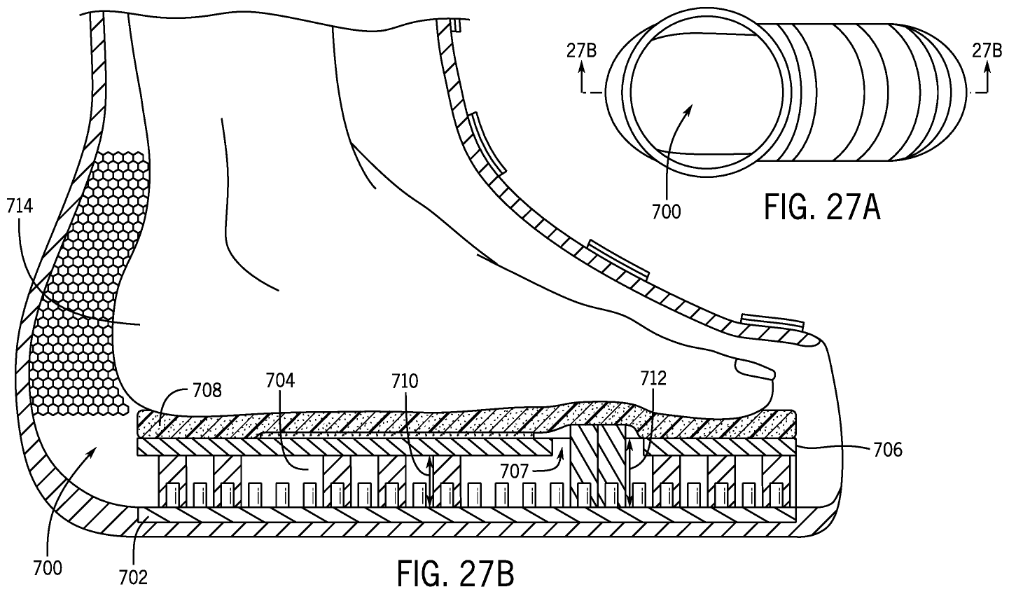

In some cases, the support columns 704 having the second axial height 712 can be replaced with biased support columns 716, which provide an upwardly directed biasing force to the region of the foot 714. FIGS. 28A and 28B show examples of a biased support columns 716. In particular, FIG. 28A shows an example of a biased support column 718, which is a specific non-limiting implementation of the biased support column 716. The biased support column 718 includes a body 720, a coupling layer 722, a fastener implemented as a bolt body 724, a recess 726, a biasing member implemented as a spring 728, and a head 730. The body 720 includes an upper surface 732, a lower surface 734, and an adjacent surface (or surfaces, such as in the case of support columns with different shapes) 736 defined between the upper surface 732 and the lower surface 734. The recess 726 is directed inwardly into the upper surface 732 of the body 720, and receives a portion of the spring 728. The spring 728 is coupled within the recess 726 at one end, and is coupled to the head 730 at the other end. The spring 728 can be sized, and can have various spring constants, desired for the specific condition (e.g., a condition that requires loading or pressure to a region of the foot 714). The bolt body 724 extends from the lower surface 734 of the body 724 and includes threads so as to threadingly engage a threaded bore of the base or sole (e.g., the biased support column 718 being removably coupled to the base or the sole). In some cases, a top surface of the head 730 (which can be formed out of the same material as the support columns) can include the coupling layer 722, which is configured to allow the biased support column 718 to be coupled to the cushioning layer 708.

FIG. 28B shows an example of another biased support column 738, which is a specific non-limiting implementation of the biased support columns 716. The biased support column 738 includes a body 740, a coupling layer 742, a first recess 745, a second recess 746, a biasing member implemented as a spring 748, and a head 750. The body 740 includes an upper surface 743, a lower surface 744, and an adjacent surface (or surfaces, such as in the case of support columns with different shapes) 746 defined between the upper surface 743 and the lower surface 744. The first recess 745 is directed inwardly into the upper surface 743 of the body 740, and receives a portion of the spring 748. The spring 748 is coupled within the recess 726 at one end, and is coupled to the head 750 at the other end. The spring 748 can be sized, and can have various spring constants, desired for the specific condition (e.g., a condition that requires loading or pressure to a region of the foot 714). The second recess 746 is inwardly directed into the lower surface 734 of the body 724, and is configured to allow for the biased support column 738 to be removably coupled to the base or sole (e.g., via pegs). In other cases, the second recess 746 can be replaced with the previously discussed protrusion so as to allow for the biased support column 738 to be removably coupled to the base or sole (e.g., via the recess in the base or the sole). In some cases, a top surface of the head 750 (which can be formed out of the same material as the support columns) can include the coupling layer 742, which is configured to allow the biased support column 738 to be coupled to the cushioning layer 708.

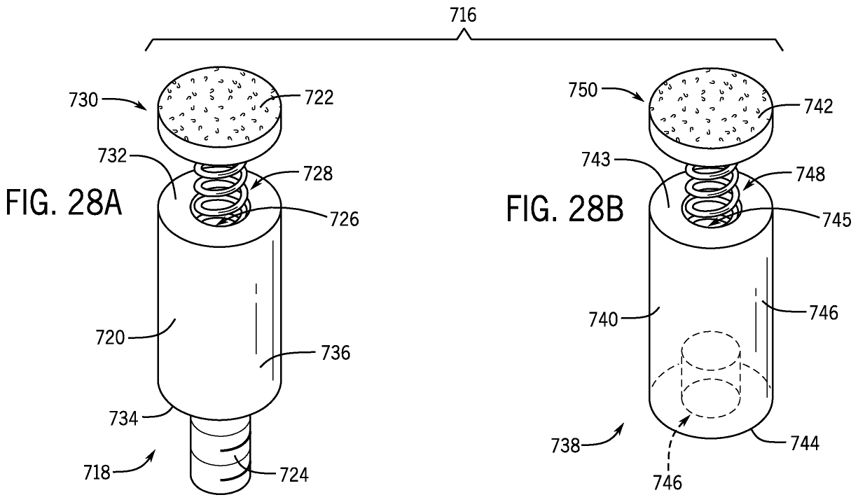

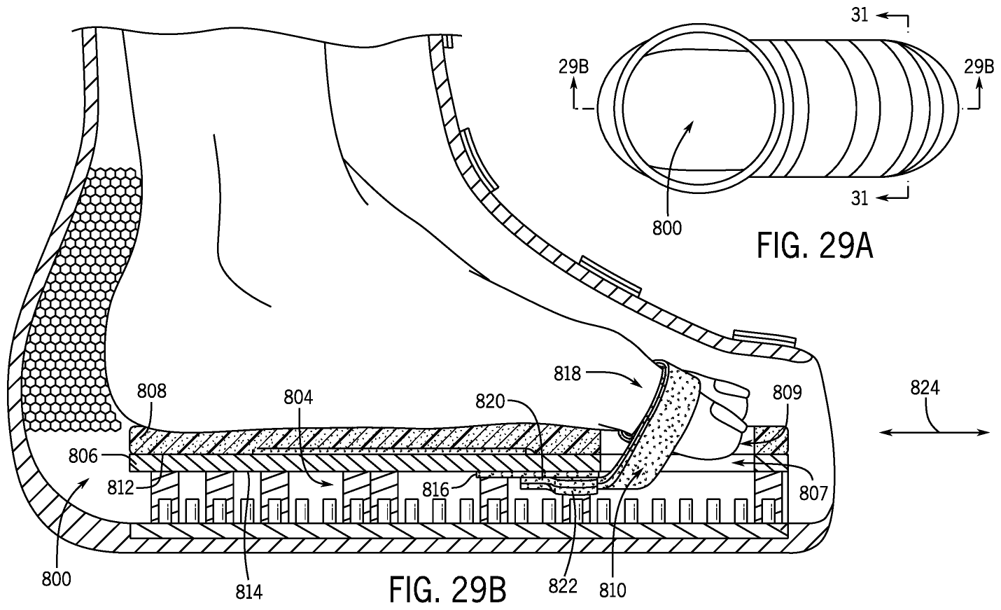

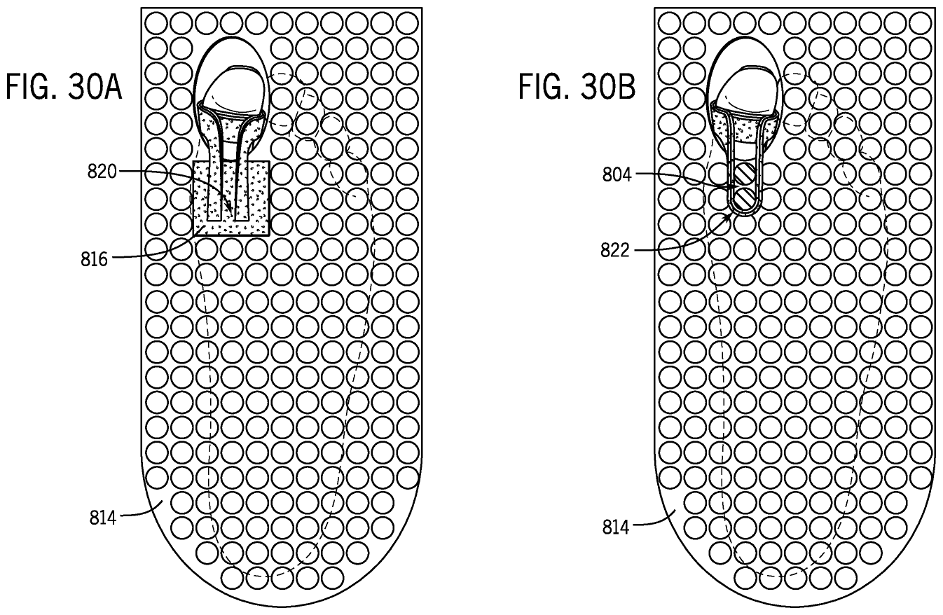

FIGS. 29A, 29B, 30A, 30B, and 31 shows an example of another example of another orthopedic insert 800 installed within footwear (e.g., the orthopedic boot 290), according to some embodiments of the disclosure. Similarly to the other previously described orthopedic inserts, the orthopedic insert 800 includes a base 802, support columns 804, an insole 806, an insole aperture 807 directed through the insole 806, a cushioning layer 808, and a cushioning layer aperture 809 directed through the cushioning layer 808. The insole 806 has an upper surface 812, an opposite lower surface 814, and a coupling layer 816 coupled to the lower surface 814.

In some embodiments, a tether 810 includes a loop 818, and a first end 820 opposite the loop 818, and a second end 822 opposite the loop 818. The loop 818 is configured to be received around and be secured to a toe (e.g., the big toe) of a subject. The loop 818 can be formed in many different ways, such as being an integrally formed loop 818 of a particular size. As illustrated, the loop 818 of the tether 810 is formed by removably coupling a portion of the tether 810 to itself, such as with a hook and loop fastener. This way, the loop 818 can be adjusted so as to accommodate various sized toes from other individuals, or toes within the same individual (e.g., other toes than the big toe). The first end 820 of the tether 810 is removably coupled to the coupling layer 816 such as with a hook and loop fastener. The second end 822 is removably coupled to one of the support columns 804. In alternative embodiments, the first end 820 and the second end 822 can be coupled or connected to their respective structures with other fasteners typically used in the art (e.g., tape, adhesives, etc.), and as previously described. When the tether 810 is secured to the toe of the subject, and when the tether 810 is secured to the other structures (e.g., support columns, coupling layers), the toe is inserted through the apertures 807, 809, and the toe is downwardly angled relative to the horizontal axis 824 (e.g., where FIG. 29B is taken along the sagittal plane, and where the horizontal axis 824 is overlaid on the sagittal plane).

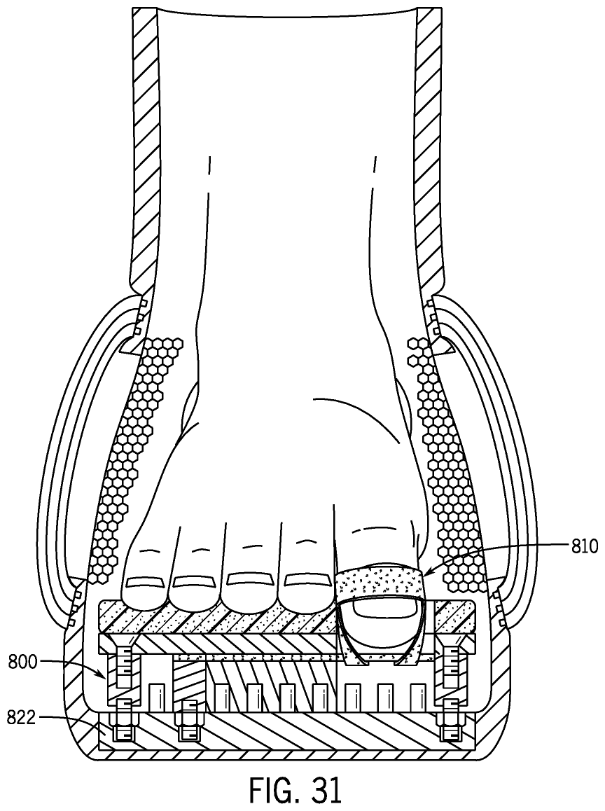

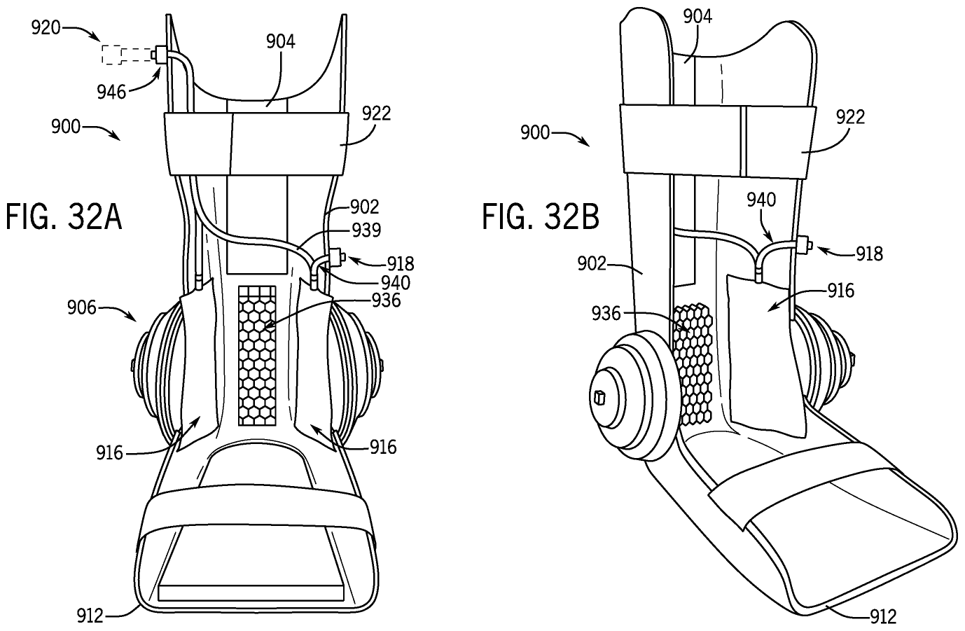

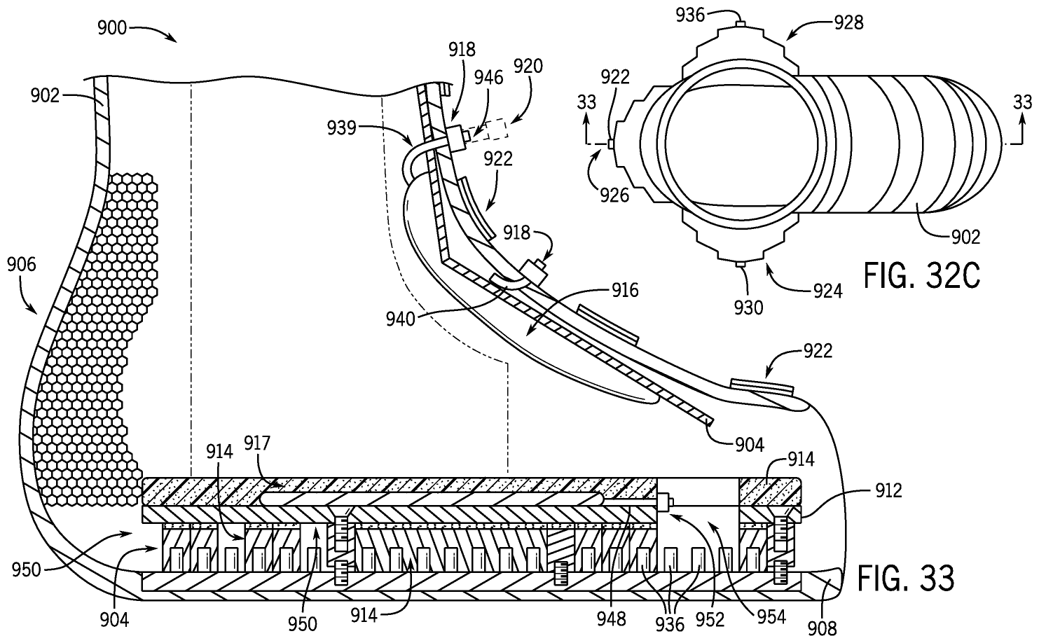

FIGS. 32A, 32B, 32C, 33, and 34 show an example of an orthopedic boot 900, which is a specific non-limiting implementation of the orthopedic boot 100. For example, the orthopedic boot 900 also includes a support member 902, supporting plates 904, an expandable shell 906, an insole 908, an insole aperture 909, a cushioning layer 910, a cushioning layer aperture 911, a sole 912, support columns 914, bladders 916, a deflate button 918, a pump 920, and fastening assemblies 922.

In some embodiments, such as shown, the expandable shell 906 is connected to and interfaces with the support member 902. In some embodiments, a periphery of the expandable shell 906 is connected to (e.g., by fasteners, stitches, adhesive, etc.) an aperture formed in the support member 902 to create an interface between the support member 902 and the expandable shell 906. In some cases, the expandable shell 906 has a single expandable domain, whereas in other cases, such as the illustrated embodiment of FIGS. 32A and 32B, the expandable shell 906 has expandable domains 924, 926, 928. The expandable domains 924, 926, 928 have corresponding tabs 930, 932, 934. The tabs 930, 932, 934 are coupled to, connected to, or integrally formed with the corresponding expandable domain. The expandable domains 924, 926, 928 are strategically situated so as to envelop a particular region of the foot of the subject prone to pressure sore formation. For example, the expandable domain 924 can be configured to surround the lateral malleolus of the subject, the expandable domain 926 can be configured to surround the heel of the subject (e.g., a portion of the calcaneus), and the expandable domain 928 can be configured to surround the medial malleolus of the subject.

In some embodiments, the expandable shell 906 (including the expandable domains 924, 926, 928) can be selectively expanded or retracted to increase or decrease the internal volume defined within a given expandable domain (or the entire expandable shell). This way, the expandable shell 906 can adjust how the expandable shell 906 contacts the foot of the subject (e.g., completely removing contact between the expandable shell or expandable domain and the foot of the subject). As shown, each expandable domain 924, 926, 928, has three sections. Each section is joined to an adjacent region by ridges, such that each of the three sections can be selectively expanded to increase or decrease the internal volume within the given expandable domain. For example, when all three sections are retracted, the internal volume within the expandable domain is the smallest, when all three sections are expanded, the internal volume within the expandable domain is the largest, and when some sections are expanded while others are retracted, the volume within the expandable domain is between the largest and smallest internal volume. Each domain is illustrated as having three sections, however in alternative embodiments, other numbers of sections could be used.

In some embodiments, a number of cushioned pads 936 (e.g., the cushioned pads 310) can be situated within the internal volume of respective expandable domains 924, 926, 928. Additionally, liner(s) 938, which can be formed of a soft fabric or foam material (e.g., a neoprene sleeve), can be positioned near against the cushioned pads 936, such that the cushioned pads 936 are positioned between the liner 938 and the expandable shell 906 (e.g., the specific expandable domains 924, 926, 928). In some embodiments, the liner(s) 938 provide additional support for the foot of the subject.

As shown, bladders 916 are positioned within the internal volume of the orthopedic boot 900 on adjacent lateral sides of the support member 902. The bladders 916 are configured to selectively expand and retract, based on a source fluid (e.g., air) being received within the bladder 916. The bladder 916 includes a conduit 939 in fluid communication with the interior of the bladder 916, and coupled to the support member 902 (e.g., via a valve 946). The valve 946 can be configured to receive a source of fluid from a pump 920 (e.g., via the valve 946) and prove the source of fluid to the bladder 916 thereby inflating the bladder 916. In some configurations, the valve 946 can be structured as a one-way valve to prevent fluid from escaping out of the bladder 916, while allowing fluid to enter the bladder 916 (e.g., from the pump 920). Another conduit 940 can be connected to the bladder 916 and provide fluid communication between the bladder 916 and the deflate button 918 (e.g., configured as a one-way valve). The deflate button 918, when depressed, allows for fluid communication between the bladder 116 and the ambient environment. In other words, if the bladder 916 is inflated, and the deflate button 918 is depressed, fluid will flow from the bladder 916 and into the ambient environment.

In some configurations, a bladder 917, which is a specific non-limiting implementation of the bladder 916, can be positioned on, connected to, or positioned underneath the cushioning layer 910. The bladder 917 can include a conduit 948 that is routed into the interior volume 950 defined between the insole 908 and the sole 912 (or base in a configuration that includes an orthopedic insert). The conduit provides fluid communication between the interior volume of the bladder 917 and the interior volume 950. In some configurations, a valve 952 can be positioned in a flow path between the conduit 948 and the interior volume 950. The bladder 917 can be inflated, and when a foot of a subject provides a force onto the bladder 917 (e.g., such as when stepping down), the bladder 917 deflates and directs air through the conduit 948, through the valve 952, and into the interior volume 950. In some cases, the valve 952 (and the conduit 948) can be positioned within a void volume 954 defined by an upper surface of the sole 912, the insole aperture 909, and the cushioning layer 910 (or other combinations). This way, a pressure sore, or region prone to pressure sore formation, received in the void volume 954 can receive a flow of fluid directed by the bladder 917. In some cases, the valve 952 can be configured (e.g., as a two-way valve), such that when the force is removed from the bladder 917 (e.g., such as when stepping up, when the a portion of the foot does not contact the bladder 917) the bladder 917 inflates (e.g., by fluid flowing upwardly through the conduit 948, through the valve 952, and into the interior volume of the bladder 917. In some cases, apertures 955, or valves 952 can be placed through the support member 902, and provide fluid communication between the ambient environment and the interior volume 950. In this case, air can readily flow between the ambient environment and the interior volume 950, which may mitigate pressure sore formation, or may accelerate healing of pressures sores. In some cases, the valves 952 as discussed above can be two-way valves. This way, when the support columns 914 or the insole 908 is compressed a fluid (e.g., air) contained within the interior volume 950 is displaced through the valve 952 and into the ambient environment (e.g., when there is some compliance for the insole 908 and the support columns 914). Additionally, when the compression loading is removed from the support columns 914 or the insole 908, a fluid flows from the ambient environment through the valve 952 and into the interior volume 950. The flowing of fluid into and out of the interior volume 950 may mitigate pressure sore formation, or may accelerate healing of pressures sores.

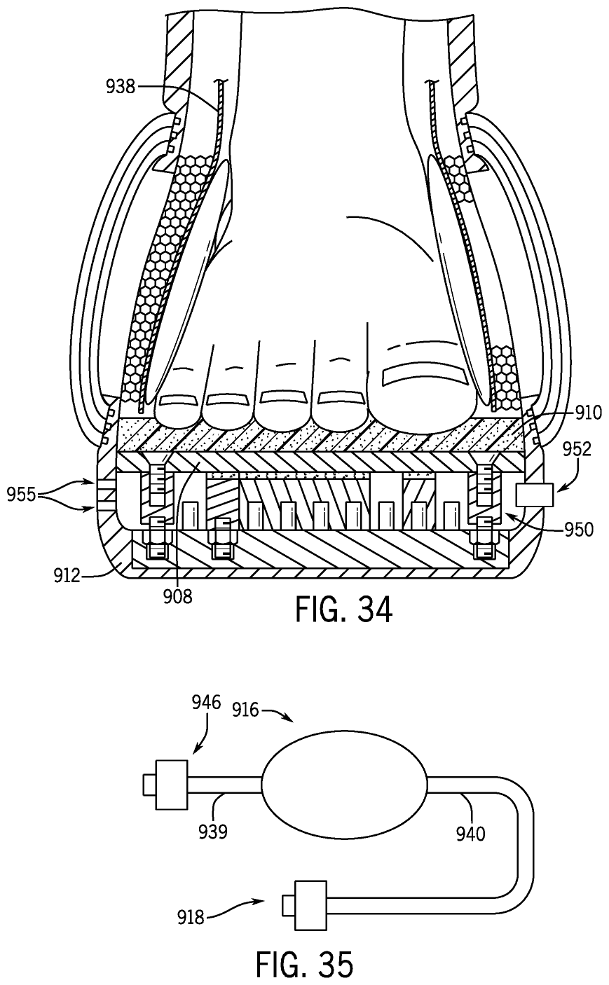

FIG. 35 shows an example of the bladder 916, according to some embodiments of the disclosure. The deflate button 618 can be configured to, when depressed (or twisted in some cases), to allow air to vent from the bladder 916 through the conduit 940 and into the ambient environment.

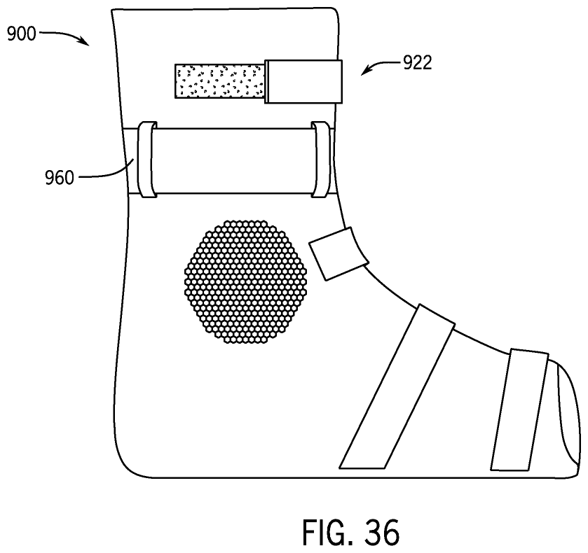

FIG. 36 shows an example of an implementation of a fastening assembly 922, which is a ring 960 surrounding and encapsulating the support member 902. The ring 960 can prevent a subject from removing the orthopedic boot 900 (or shoe). The ring 960 can be formed of rigid materials, such as fiberglass, plastic, metal, etc.

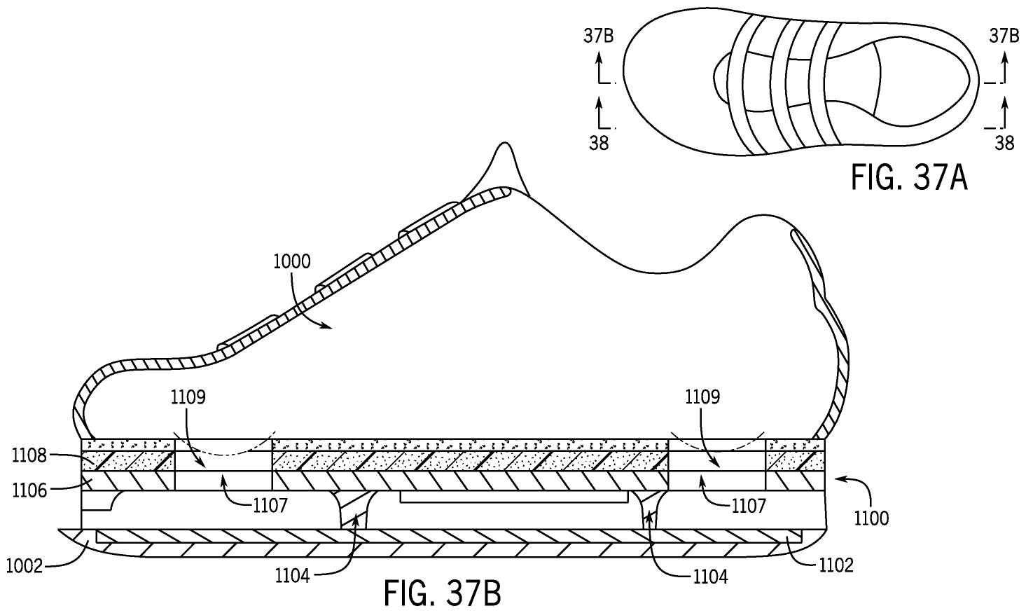

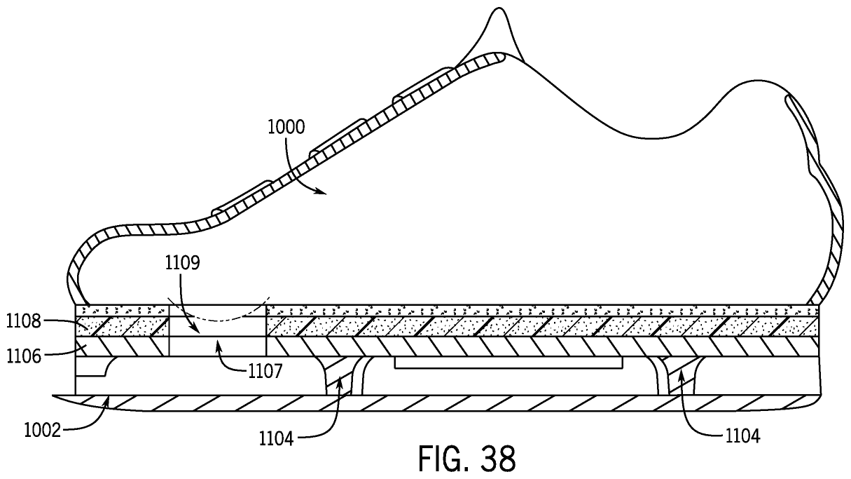

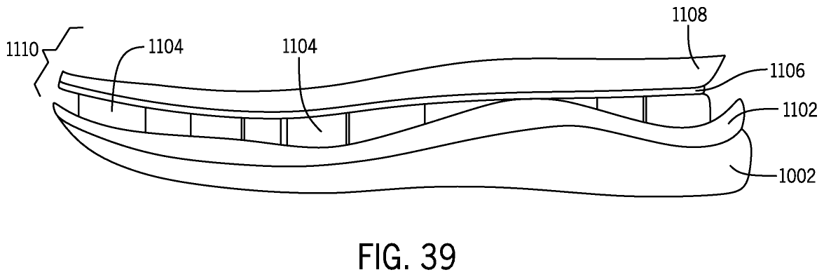

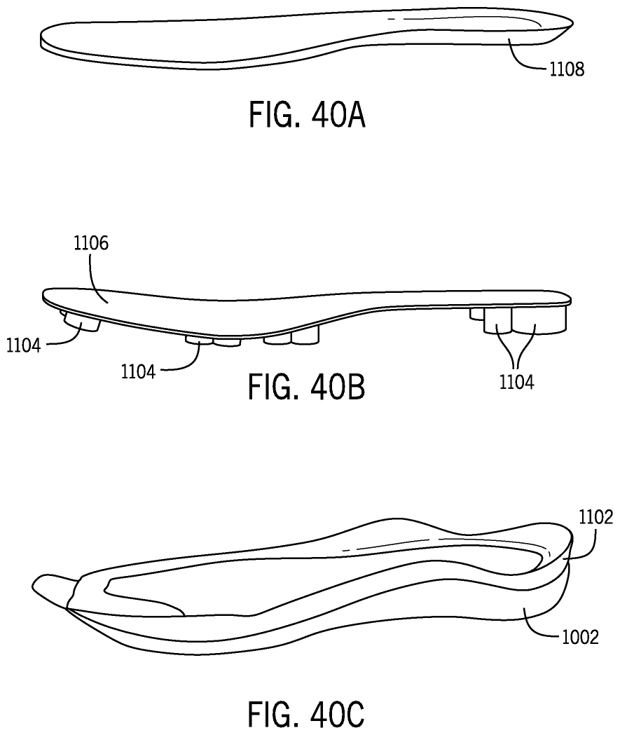

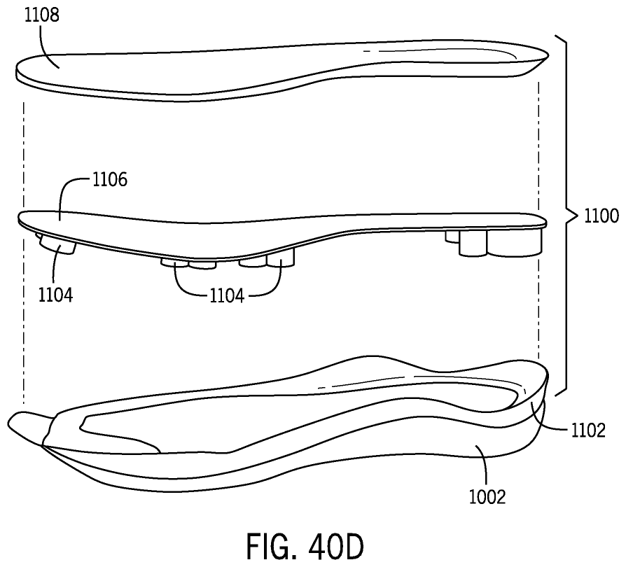

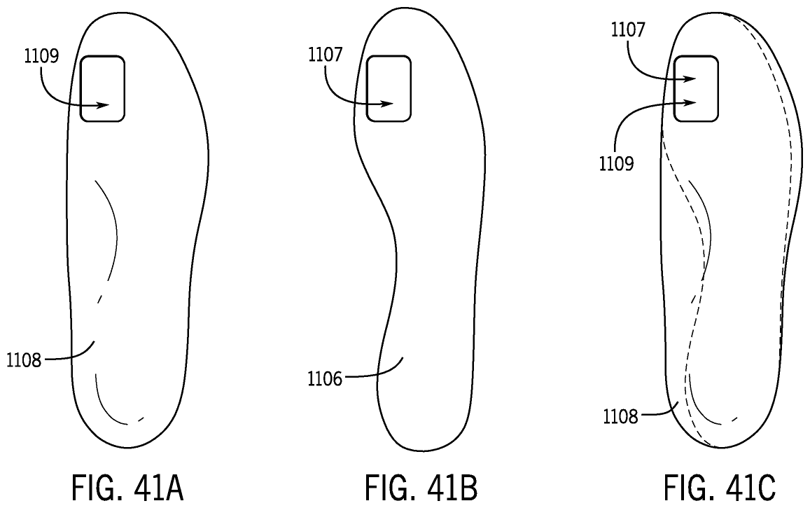

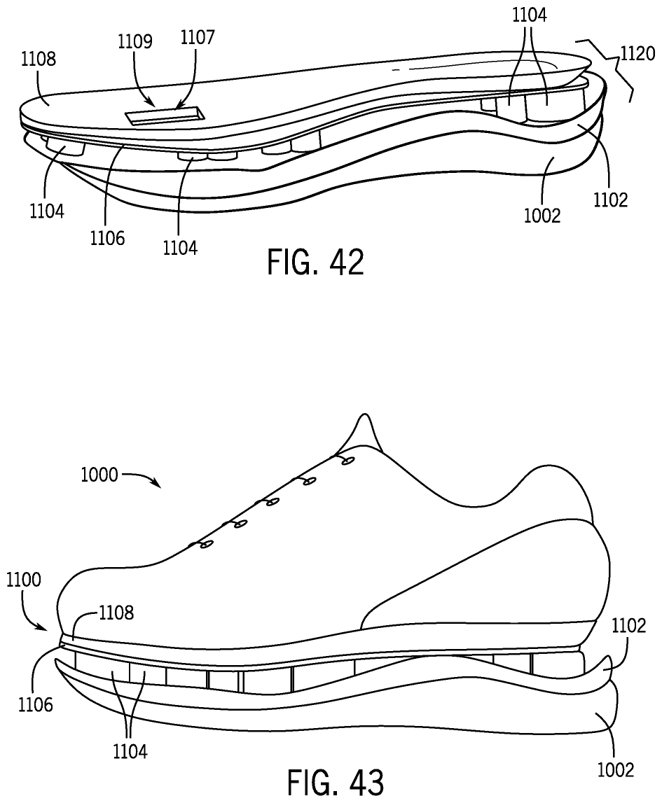

FIGS. 37A-43 show an example of an implementation of a shoe 1000 having an orthopedic insert 1100 disposed within the shoe 1000. The orthopedic insert 1100 is similar to the previously discussed orthopedic inserts. For example, the orthopedic insert 1100 also includes a base 1102, support columns 1104, an insole 1106, an insole aperture 1107 directed through the insole 1106, a cushioning layer 1108, and a cushioning layer aperture 1109 directed through the cushioning layer 1108. As shown, the base 1102 is connected to the support columns 1104, the support columns 1104 are connected to the insole 1106, the insole 1106 is connected to the cushion cushioning layer 1108. Each of these components can be removably coupled to each other rather than connected (e.g., the insole 1106 is removably coupled to the cushioning layer 1108). The support columns 1104 can be removably coupled to the base 1102 and the insole 1106. The support columns 1104 can have varying shapes and sizes, particularly the axial height of the support columns 1104. For example, FIG. 42 shows the axial heights of the support columns 1104 vary to contour the shape (e.g., the topology) of the base 1102, where the base 1102 contours the shape and interfaces with the sole 1002 of the shoe 1000. In other configurations, the base 1102 can be omitted and the axial height of the support columns 1104 can contour the topology of the sole 1002 of the shoe 1000. In either configuration, the insole 1106 being supported by the support columns 1104 is maintained substantially parallel to a walking surface (e.g., the ground).

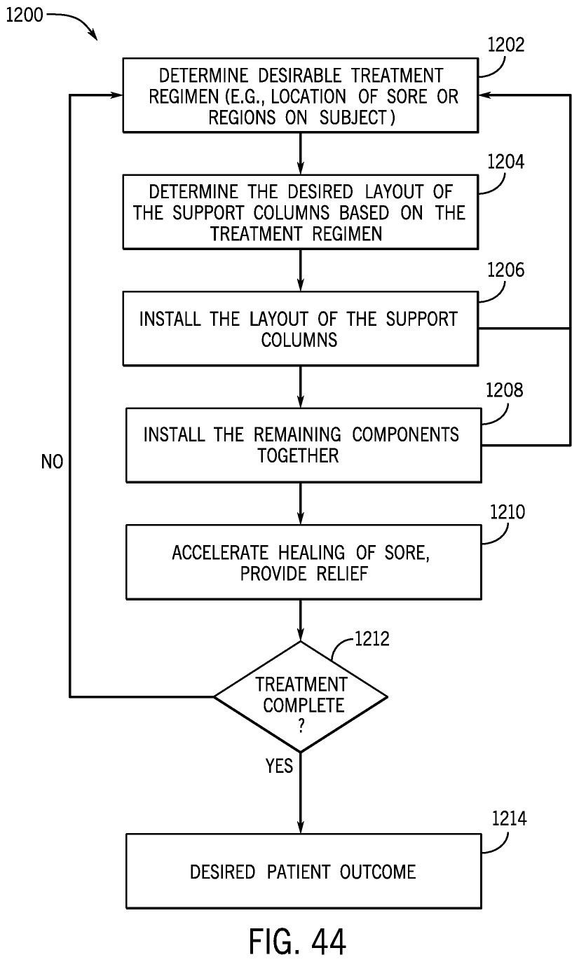

FIG. 44 shows an example of flowchart of a process 1200 for treating or preventing the formation of pressure sores (e.g., wounds, ulcers, etc.). At 1202, process 1200 can include determining the desirable treatment regimen, which can include determining locations to avoid applying pressure (or removing an amount of pressure from), such as already formed pressure sores, wounds, scabs, regions prone to pressure sore formation, etc. In some cases, pressure sensor arrays 1110 (e.g., two-dimensional pressure sensors) can be placed to determine a loading profile of a foot of a subject. For example, the pressure sensor array 1110 alone can be placed on the ground and a foot of a subject is placed on the pressure sensor array. Then, three-dimensional pressure data can be extracted from the pressure sensor array 1110 (e.g., using a suitable computing device, such as a computer, etc., in communication with the pressure sensor array 1110). The three dimensional pressure data can be displayed and can be used to direct a practitioner (e.g., a technician, a doctor, etc.) to determine a desired loading profile for creating an orthopedic insert (or shoe, or other footwear), according to some embodiments of the disclosure. In some cases, the created orthopedic insert (or other footwear) can be universal, so as to avoid applying pressure to typically aggravated areas, such as areas prone to pressure sore formation (e.g., in some cases, ulcers frequently develop over bony prominences, such as the heel or malleolus).

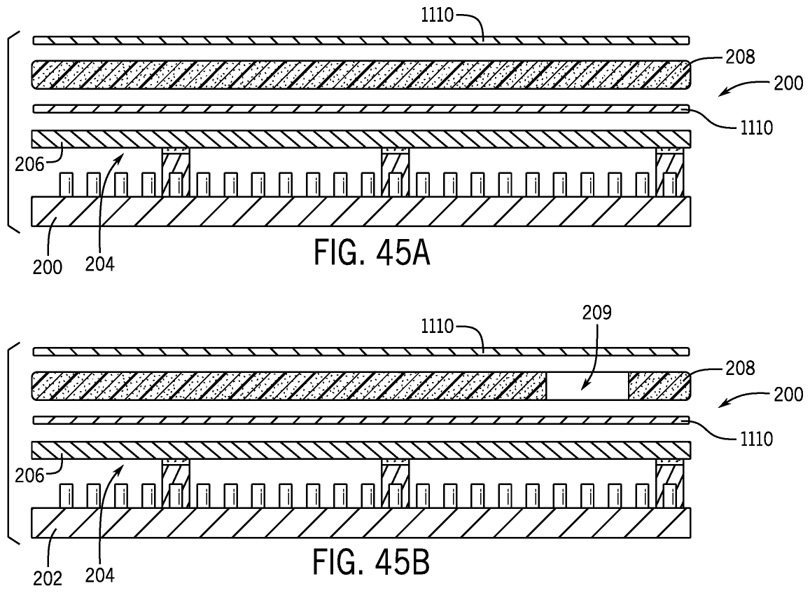

At 1204, process 1200 can include determining the desired layout of the support columns, and types of support columns, based on the treatment regimen. For example, areas that have been defined as desired to be unloaded (or loaded in the case of conditions such as metatarsaglia), are identified and the support columns are installed at 1206 of process 1200 (or removed) to create the desired configuration. For example, unloaded regions do not have support columns, whereas loaded regions have support columns. In some cases at 1206, process 1200 can revert back to determining the desired configuration by utilizing the pressure sensor array 1110. For example, FIG. 45A shows an example of pressure sensor arrays 1110 disposed between the cushioning layer 208 of the orthopedic insert 200. The pressure data captured from the pressure sensors arrays 1100 can be used to readjust the number and type of support columns installed as desired.

At 1208, process 1200 can include installing the remaining components of the orthopedic insert or other footwear, which can include forming the apertures (e.g., by stamping, cutting, using a cast saw, using sanders, etc.) in the insole and the cushioning layer. In some embodiments, the components of the orthopedic insert (or orthopedic boot) can be trimmed accordingly (e.g., by stamping, cutting, using a cast saw, using sanders, etc.). Similarly to above, the pressure sensor arrays 1100 can be used to determine the loading after apertures have been formed in either or both of the insole and the cushioning layer. FIG. 45B shows the pressure sensor arrays 1100 being used to test the loading profile after the cushioning layer aperture has been formed. After the desired pressure profile or loading profile on the foot is implemented with the orthopedic insert or footwear, at 1210, process 1200 can include the acceleration of healing of already formed pressure sores, or the prevention of formation of pressure sores.

At 1212, process 1200 can determine whether or not treatment is complete. For example, in some cases, such as when the subject receives a routine checkup, a practitioner may check or determine that the orthopedic insert or footwear needs to be adjusted, and thus the process 1200 can begin back at 1202. Alternatively, the practitioner may determine that all of the pressure sores have healed, or no pressure sores have formed (e.g., after finishing a treatment regimen of a cast), and thus the process 1200 may proceed to and be completed at 1214 where the patient has a desired patient outcome.

It should be understood that the above described steps of the process of FIG. 44 can be executed or performed in any order or sequence not limited to the order and sequence shown and described in the figures. Also, some of the above steps of the process of FIG. 44 can be executed or performed substantially simultaneously where appropriate.

Although the invention has been described and illustrated in the foregoing illustrative embodiments, it is understood that the present disclosure has been made only by way of example, and that numerous changes in the details of implementation of the invention can be made without departing from the spirit and scope of the invention, which is limited only by the claims that follow. Features of the disclosed embodiments can be combined and rearranged in various ways.

1. An orthopedic boot comprising:
a sole;
a support member connected to and upwardly extending from the sole, the support member being adapted to secure the boot to a foot of a subject; and
a plurality of support columns removably coupled to and upwardly extending from the sole.

2. The orthopedic boot of claim 1, wherein the sole has a first surface and an opposite second surface, the second surface being configured to contact the ground,
wherein a column of the plurality of columns has an upper surface,
wherein a portion of first surface of the sole and the upper surface at least partially define a void, and
wherein when a foot of a subject is secured by the support member, a sore of a subject is positioned within the void.

3. The orthopedic boot of claim 1, wherein the sole has a first surface and an opposite second surface, the second surface being configured to contact the ground,
wherein a first column of the plurality of columns has a first upper surface and a first height,
wherein a second column of the plurality of columns has a second upper surface and a second height,
wherein the first height is different than the second height, and the first surface and the second surface at least partially define a void, and
wherein when a foot of a subject is secured by the support member, a sore of a subject is positioned within the void.

4. The orthopedic boot of claim 1, further comprising an insole coupled to the plurality of support columns and positioned above the sole,
wherein the base does not contact the insole,
wherein the insole is rigid, and
wherein the sole is rigid.

5. The orthopedic boot of claim 4, further comprising an aperture extending through the insole, and wherein two support columns of the plurality of support columns are spaced apart from each other by a distance.

6. The orthopedic boot of claim 5, further comprising a second aperture extending through the insole.

7. The orthopedic boot of claim 5, wherein the sole has a first surface and an opposite second surface, the second surface being configured to contact the ground,
wherein the aperture and the first surface defines a void volume, and
wherein the plurality of support columns are not positioned within the void volume.

8. The orthopedic boot of claim 5, further comprising:
a cushioning layer coupled to the insole and positioned above the plurality of support columns, the cushioning layer comprising a memory foam, and
a cushioning layer aperture extending through the cushioning layer, and
wherein a portion of the aperture and a portion of the cushioning layer aperture overlap.

9. The orthopedic boot of claim 5, wherein when a foot of a subject having a sore is positioned on the insole, a perimeter of the aperture surrounds the sore to prevent contact between the orthopedic boot and the sore of the subject.

10. The orthopedic boot of claim 1, further comprising a base, the base being coupled to the sole, and
wherein the plurality of support columns are removably coupled to the base.

11. The orthopedic boot of claim 10, wherein the base is at least one of affixed, and removably coupled to the sole.

12. The orthopedic boot of claim 10, wherein the base has a first surface and an opposite second surface, the second surface being removably coupled to the sole, and further comprising a plurality of pegs upwardly extending from the first surface.

13. The orthopedic boot of claim 12, wherein a first support column of the plurality of support columns includes an upper surface, a lower surface, and a recess, and
wherein a given peg of the plurality of pegs is inserted into the recess to secure the first support column to the base.

14. The orthopedic boot of claim 13, wherein the upper surface of the first support column is angled relative to a horizontal axis.

15. The orthopedic boot of claim 14, wherein the upper surface of the first support column is angled downwardly in a lateral direction relative to the sole.

16. The orthopedic boot of claim 14, wherein the upper surface of the first support column is angled upwardly in a lateral direction relative to the sole.

17. The orthopedic boot of claim 14, wherein the upper surface of the first support column is angled relative to a front view of the first support column.

18. The orthopedic boot of claim 13, wherein the first support column includes the recess and a second recess, and
wherein a second peg of the plurality of pegs is inserted into the second recess.

19. The orthopedic boot of claim 18, wherein the lower surface has the recess and the second recess, and wherein an upper surface of the first support column is angled relative to a horizontal axis.

20. The orthopedic boot of claim 10, wherein a first support column of the plurality of support columns includes a first surface and a second surface, and further comprising a fastener coupled to the first support column, the fastener being configured to couple the first support column to the base.

21. The orthopedic boot of claim 20, wherein the first surface of the first support column has a bolt body extending therethough, and further comprising a bore extending through the base, and
wherein the bolt body threadingly engages the bore to couple the first support column to the bore of the base.

22. The orthopedic boot of claim 21, further comprising:
an insole coupled to the plurality of support columns and positioned above the sole; and
a second fastener, the second fastener extending through the insole and configured to engage the second surface of the first support column to secure the insole to the first support column.

23. The orthopedic boot of claim 20, wherein the first surface of the first support column has a bore extending therethrough, and further comprising a bolt body extending from the base, and
wherein the bolt body threadingly engages the bore to couple the first support column to the bolt body of the base.

24. The orthopedic boot of claim 10, wherein the base has a first surface and an opposite second surface, the base including a plurality of recesses directed into the first surface of the base.

25. The orthopedic boot of claim 24, wherein a first support column of the plurality of support columns includes a protrusion extending therefrom, and
wherein the protrusion is inserted into a given recess of the plurality of recesses to secure the first support column to the base.

26. The orthopedic boot of claim 25, wherein the given recess and the protrusion have at least one of a rectangular shape, and a round shape.

27. The orthopedic boot of claim 25, wherein the first support column includes a second protrusion extending therefrom, and
wherein a second protrusion is inserted into another recess of the plurality of recesses to secure the first support column to the base.

28. The orthopedic boot of claim 22, wherein the column includes a first surface and a second surface, the first surface including at least one of a hook fastener, and a loop fastener.

29. The orthopedic boot of claim 28, further comprising an insole coupled to the plurality of support columns and positioned above the sole, and
wherein the insole includes the other of the at least one of the hook fastener and the loop fastener to removably couple the insole to the plurality of support columns.

30. The orthopedic boot of claim 1, wherein at least one of the sole, and the base has a first surface and an opposite second surface, the first surface of the base including at least one of a hook fastener, and a loop fastener.

31. The orthopedic boot of claim 30, wherein a given column of the plurality of support columns is configured to contact an adjacent column.

32. The orthopedic boot of claim 31, wherein the plurality of columns each include an upper surface and an opposite lower surface, the lower surface having the other of the at least one of the hook fastener, and the loop fastener.

33. The orthopedic boot of claim 31, wherein each of the plurality of columns includes a side surface, and
wherein a first side surface for each column of a subset of the plurality of columns defines a boundary, the boundary defining a void volume, such that the plurality of columns are not positioned within the void volume.

34. The orthopedic boot of claim 33, wherein when a foot of a subject is secured by the support member, a sore of a subject is positioned within the void volume.

35. The orthopedic boot of claim 34, further comprising:
an insole coupled to and positioned above the plurality of support columns; and
a cushioning layer coupled to and positioned above the insole, the cushioning layer comprising memory foam.

36. The orthopedic boot of claim 30, wherein the support member has an interior surface defining an interior volume, the foot of the subject being configured to be received within the internal volume of the support member, and
further comprising a bladder received within the interior volume, the bladder being configured to receive a source of fluid thereby expanding the bladder.

37. The orthopedic boot of claim 1, further comprising an expandable shell coupled to the support member.

38. The orthopedic boot of claim 37, further comprising an aperture extending through the expandable shell.

39. The orthopedic boot of claim 37, wherein the expandable shell is positioned around a portion of a heel of a subject when a foot of the subject is received within an internal volume of the support member.

40. The orthopedic boot of claim 37, further comprising a plurality of pads situated within the expandable shell.

41. The orthopedic boot of claim 40, further comprising a liner situated within an internal volume of the support member, the plurality of pads positioned between the liner and the expandable shell.

42. The orthopedic boot of claim 1, further comprising:
an insole coupled to and positioned above the plurality of support columns;
an aperture extending through the insole; and
a tether configured to secure a toe of a subject to the insole.

43. The orthopedic boot of claim 42, wherein the tether includes a loop and an end, the end removably coupled to at least one of the insole and a first support column of the plurality of support columns by a hook and loop fastener.

44. The orthopedic boot of claim 43, wherein the loop of the tether is configured to be received around a toe of a subject, and when the end of the tether is coupled to the at least one of the insole and the first support column, the toe is secured at an angle relative to a horizontal axis.

45. The orthopedic boot of claim 1, wherein a first support column of the plurality of support columns includes a biasing member configured to exert a biasing force.

46. The orthopedic boot of claim 45, wherein the first support column includes a head, the head being coupled to the biasing member, and
wherein the biasing member is a spring.

47. The orthopedic boot of claim 1, further comprising a ring surrounding the support member, the ring configured to prevent removal of the support member from the subject.

48. The orthopedic boot of claim 1, further comprising an insole situated above and removably coupled to the plurality of support columns.

49. The orthopedic boot of claim 48, wherein a first support column of the plurality of support columns includes a first surface and a second surface, the first surface including at least one of a hook fastener, and a loop fastener, and
wherein the insole includes the other of the at least one of the hook fastener and the loop fastener to removably couple the insole to the plurality of support columns.

50. The orthopedic boot of claim 48, further comprising a cushioning layer, the cushioning layer comprising a memory foam, and
wherein the cushioning layer is situated above and coupled to the insole.

51. The orthopedic boot of claim 50, wherein the cushioning layer is removably coupled to the insole.

52. An orthopedic insert for use with an orthopedic boot or a shoe, the orthopedic insert comprising:
a base;
a plurality of support columns upwardly extending from and removably coupled to the base;
an insole coupled to the plurality of support columns, the insole positioned above the base, and
wherein the orthopedic insert is configured to be received within an internal volume of the orthopedic boot or the shoe.

53. The orthopedic insert of claim 52, further comprising an aperture extending through the insole, and wherein the aperture and a surface of the base define a void volume.

54. The orthopedic insert of claim 53, wherein the plurality of support columns are not positioned within the void volume.

55. The orthopedic insert of claim 53, wherein when a foot of a subject is positioned on the insole, a sore of a subject is positioned within the void volume.

56. The orthopedic insert of claim 52, further comprising a cushioning layer coupled to a situated above the insole, the cushioning layer comprising memory foam.
